(19) United States
(12) Patent Application Publication
(10) Pub. No.: US 2025/0297893 A1
(43) Pub. Date: Sep. 25, 2025

(54) DEVICES, METHODS, AND SYSTEMS FOR IMAGING, SENSING, MEASURING AND RECORDING SPECTRUM

(71) Applicant: SENSILL, INC., Concord, CA (US)

(72) Inventors: Yan Zhou, Pleasanton, CA (US); Richard Wyeth, Granite Bay, CA (US); William Shea, Martinez, CA (US)

(73) Assignee: SENSILL, INC., Concord, CA (US)

(21) Appl. No.: 19/229,731

(22) Filed: Jun. 5, 2025

Related U.S. Application Data
(63) Continuation of application No. PCT/US2023/083068, filed on Dec. 8, 2023.
(60) Provisional application No. 63/431,507, filed on Dec. 9, 2022.
(60) Provisional application No. 63/431,510, filed on Dec. 9, 2022.
(60) Provisional application No. 63/431,519, filed on Dec. 9, 2022.
(60) Provisional application No. 63/431,525, filed on Dec. 9, 2022.
(60) Provisional application No. 63/431,528, filed on Dec. 9, 2022.
(60) Provisional application No. 63/431,533, filed on Dec. 9, 2022.

(51) Int. Cl. G01J 3/453; G01J 3/02; G01J 3/04; G01J 3/14

(52) U.S. Cl. CPC G01J 3/453; G01J 3/0208; G01J 3/0224; G01J 3/04; G01J 3/14

(57) ABSTRACT

Devices, systems, and methods include a system for analyzing a target area. The system may include an opaque member having one or more slits configured to be transverse to a target area, one or more lenses configured to receive light from the target area, and an image sensor configured to receive the light from the target area that has passed through the one or more slits and the one or more lenses. A controller may be in communication with the image sensor to process and/or monitor sensed light. The system may include an optical system that includes the opaque member and the one or more lenses, which may have a first set of lenses and a second set of lenses. The target area may include a substrate supporting one or more reactants. The system may be a Fourier transform hyperspectral imaging fluid analysis system.

### CROSS-REFERENCE TO RELATED APPLICATIONS

This application is a continuation of International Application No. PCT/US2023/083068, filed Dec. 8, 2023, which claims priority to: U.S. Provisional Patent Application Ser. No. 63/431,507, filed Dec. 9, 2022, the entirety of which is incorporated herein by reference; U.S. Provisional Patent Application Ser. No. 63/431,510, filed Dec. 9, 2022, the entirety of which is incorporated herein by reference; U.S. Provisional Patent Application Ser. No. 63/431,519, filed Dec. 9, 2022, the entirety of which is incorporated herein by reference; U.S. Provisional Patent Application Ser. No. 63/431,525, filed Dec. 9, 2022, the entirety of which are incorporated herein by reference; U.S. Provisional Patent Application Ser. No. 63/431,528, filed Dec. 9, 2022, the entirety of which are incorporated herein by reference; U.S. Provisional Patent Application Ser. No. 63/431,533, filed Dec. 9, 2022, the entirety of which are incorporated herein by reference.

### TECHNICAL FIELD

The present disclosure pertains to sensing and analysis tools, and the like. More particularly, the present disclosure pertains to devices and systems for imaging, sensing, measuring, and recording light from a target area, and methods for manufacturing and using such devices and systems.

### BACKGROUND

A wide variety of devices have been developed for collection, storing, sensing, and analysis of light from target areas. These devices are manufactured by any one of a variety of different manufacturing methods and may be used according to any one of a variety of methods. Of the known medical devices and methods, each has certain advantages and disadvantages.

### BRIEF SUMMARY

This disclosure provides design, material, manufacturing method, and use alternatives for sensing and analysis devices. Although it is noted that collection, storing, sensing, and analysis approaches and systems are known, there exists a need for improvement on those approaches and systems.

An example system including a substrate, one or more reactants on the substrate, an opaque member having a first slit and/or a second slit, and an image sensor configured to receive light reflected or scattered or remitted from the one or more reactants and pass through the first slit and the second slit.

Alternatively or additionally to any of the embodiments in this section, the system may further include a cylinder lens and an imaging lens positioned between the substrate and the opaque member.

Alternatively or additionally to any of the embodiments in this section, the system may further include a spherical or aspheric lens and a cylinder lens positioned between the opaque member and the image sensor.

Alternatively or additionally to any of the embodiments in this section, the system may further include a third slit in front of the first slit and the second slit, wherein third slit is through an opaque member spaced from the opaque member having the first slide and the second slide and toward the substrate.

In another example, a system for analyzing a target area may include an opaque member having one or more slits configured to be transverse to the target area, one or more lenses configured to receive light from the target area, and an image sensor configured to receive the light from the target area that has passed through the one or more slits and the one or more lenses.

Alternatively or additionally to any of the embodiments in this section, the one or more slits may include a first slit and a second slit parallel to and spaced from the first slit.

Alternatively or additionally to any of the embodiments in this section, the one or more lenses may include a focusing lens and an imaging lens configured to receive the light from the target area prior to the light passing through the one or more slits.

Alternatively or additionally to any of the embodiments in this section, the one or more lenses may include a focusing lens and an imaging lens configured to receive the light from the target area after the light has passed through the one or more slits and before the light reaches the image sensor.

Alternatively or additionally to any of the embodiments in this section, the one or more lenses may include a first set of one or more lenses configured to receive the light from the target area prior to the light passing through the one or more slits and a second set of one or more lenses configured to receive the light from the target area after the light has passed through the one or more slits and before the light reaches the image sensor.

Alternatively or additionally to any of the embodiments in this section, the system may further include an interferometer configured to receive the light from the target area after the light has passed through the one or more slits and before the light reaches the image sensor.

Alternatively or additionally to any of the embodiments in this section, the interferometer may include one or more beam splitter/combiners, a first mirrored surface, and a second mirrored surface.

Alternatively or additionally to any of the embodiments in this section, the first mirrored surface may be non-perpendicular relative to the second mirrored surface.

Alternatively or additionally to any of the embodiments in this section, the interferometer may include a prism having a first total internal reflection surface and a second total internal reflection surface.

Alternatively or additionally to any of the embodiments in this section, the interferometer may include a first polarizer, a second polarizer, and a beam splitter positioned between the first polarizer and the second polarizer.

Alternatively or additionally to any of the embodiments in this section, the target area may include an array of reactants, the light received at the one or more lenses and the image sensor is light from the array of reactants, and the system may further include a substrate supporting the array of reactants and a controller in communication with the image sensor, wherein the controller may be configured to identify a component of fluid in contact with the array of reactants based on the light from the array of reactants received at the image sensor.

In another example, an optical system for use in a fluid analysis system, the optical system may include a first set of lenses, a second set of lenses, an opaque member having one or more slits therein and positioned between the first set of lenses and the second set of lenses, wherein the first set of lenses may be configured to form an image of an array of reactants at the opaque member and the second set of lenses are configured to form an interferogram from light passing through the one or more slits on a surface.

Alternatively or additionally to any of the embodiments in this section, the one or more slits may comprise a first slit and a second slit parallel to and spaced from the first slit.

Alternatively or additionally to any of the embodiments in this section, the first set of lenses may comprise one or both of a focusing lens and an imaging lens configured to receive light from the array of reactants to form the image of the array of reactants on the opaque member.

Alternatively or additionally to any of the embodiments in this section, the second set of lenses comprise one or both of a focusing lens and an imaging lens configured to form the interferogram on the surface.

Alternatively or additionally to any of the embodiments in this section, the system may further include an interferometer configured to receive light from the array of reactants after the light has passed through the one or more slits.

Alternatively or additionally to any of the embodiments in this section, the system may further include a housing configured to house the first set of lenses, the second set of lenses, and the opaque member.

In another example, a hyperspectral imaging fluid analysis system may include a substrate, one or more reactants supported by the substrate, an opaque member having one or more slits, an image sensor configured to receive light from the one or more reactants that has passed through the one or more slits, and a controller in communication with the image sensor.

Alternatively or additionally to any of the embodiments in this section, the controller may be configured to identify a component of fluid in contact with the one or more reactants based on the light from the one or more reactants received at the image sensor.

Alternatively or additionally to any of the embodiments in this section, the one or more slits may include a first slit and a second slit.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic diagram of an illustrative sensing system;

FIG. 2 is a schematic diagram of an illustrative sensing system;

FIG. 3 is a schematic diagram of an illustrative computing system;

FIG. 4 is a schematic diagram of an illustrative optical system;

FIGS. 5A and 5B are side and top views, respectively, of an illustrative sensing system;

FIG. 6 is a schematic top view of an illustrative optical system;

FIGS. 7A-7C are schematic views an illustrative optical system;

FIG. 8 is a schematic top view of an illustrative optical system;

FIG. 9 is a schematic diagram of an illustrative optical system utilizing an interferometer;

FIGS. 10A and 10B are schematic side and top views, respectively, of an illustrative sensing system utilizing an interferometer;

FIG. 11 is a schematic view of an illustrative sensing system utilizing an interferometer;

FIG. 12 is a schematic view of an illustrative sensing system utilizing an interferometer;

FIG. 13 is a schematic view of an illustrative sensing system utilizing an interferometer;

FIG. 14 is a schematic view of an illustrative sensing system utilizing an interferometer;

FIG. 15 is a schematic view of an illustrative sensing system utilizing an interferometer;

FIG. 16 is a schematic view of an illustrative sensing system utilizing an interferometer; and

FIG. 17 is a schematic view of an illustrative technique for analyzing a target area.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

The term “fluid” is inclusive of both liquids and gases.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “a configuration”, “some configurations”, “other configurations”, etc., indicate that the configuration described may include one or more particular features, structures, and/or characteristics. However, such recitations do not necessarily mean that all embodiments include the particular features, structures, and/or characteristics. Additionally, when particular features, structures, and/or characteristics are described in connection with one configuration, it should be understood that such features, structures, and/or characteristics may also be used in connection with other configurations whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure. Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

Fluids with concentrations of volatile compounds (e.g., volatile organic compounds (VOCs)) and/or gasses, which may or may not be hazardous, may be sensed, analyzed, and/or monitored. Sensing, analyzing, and/or monitoring of fluids with analytes (e.g., non-volatile and/or volatile compounds, gases, liquids, and/or other fluids) may utilize absorption measurements of reactants exposed to such fluids for any purpose including, but not limited to, diagnostic hazard warning, manufacturing processes or quality control, record keeping, archival purposes, product development, product-consumer matching, etc.

In some cases, VOCs and/or gasses may be present in ambient fluid (e.g., ambient air, etc.) and sensed, analyzed, and/or monitored using reactants for real-time alarms, to treat subjects, or to collect and/or archive data for health records, regulatory compliance records, etc. Further, VOCs and/or gasses exhaled or emitted, excreted, emanated, released, and/or secreted from a subject (e.g., humans, animals other than humans, food, produce, meat, pathogens, bacteria (e.g., good and/or bad bacteria), plants, wounds, ulcers, surgical sites, skin of a subject, mouth of a subject, nasal passages of a subject, sinuses of a subject, rectum area of a subject, vaginal area of a subject, genitals area of a subject, ear canals of a subject, pores of a subject, etc.) may be sensed, analyzed, and/or monitored to assess hazardous, dangerous, or illegal substances in or at the subject or target site, a lung condition of lungs of a subject, a condition of a blood disease, a condition of infections, conditions related to diseases or biological conditions, conditions related to general health, conditions related to food flavors, conditions related to perfumes or smells, and/or other suitable conditions.

The systems discussed herein for sensing, analyzing, and/or monitoring targets (e.g., fluids with analytes of interest and/or other suitable targets) may be configured to accurately detect and record changes over time at a target area. In one example, the system discussed herein may be configured to sense, analyze, and/or monitor fluids by accurately detecting and recording one or more colorimetric sensor arrays (CSAs) spectral response to exposure to the fluids. The systems may utilize techniques for non-invasively detecting analytes of interest (e.g., one or more pathogens responsible for specific human skin infections including, but not limited to, skin infections, urinary tract infections (UTIs), vaginitis, wound infections, ulcers, etc., and/or other suitable analytes) from a fluid using a CSA to allow for early detection of and early implementation of protocols to address one or more conditions associated with any sensed analytes of interest. In one example, enhanced classification of one or more analytes using the systems described herein may enable detection and identification of responsible pathogens at the very beginning stages of a dangerous skin infection, which may result in a high level of protection and probability of a favorable outcome for subjects.

The systems for sensing, analyzing, and/or monitoring targets use optics to capture photons diffused, reflected, scattered, transmitted, or reemitted from the targets. In some examples, a system configured to sense, analyze, and/or monitor a target comprising analytes of interest in fluids may use optics to capture photons diffused, reflected, scattered, transmitted, or reemitted from individual reactants (e.g., color areas, color imprints, color bars, color dots, etc.) applied to a substrate or membrane of a CSA and deliver the photons via a fiber optic cable or free space optics to a light collector (e.g., a high-resolution spectrometer having a photodetector and/or other suitable light collector) for measurement of collected light. Appropriate calibration techniques and an algebraic signal processing algorithm may be applied to the measurements to calculate a light collection measurement (e.g., reflectivity, intensity, pixel value, photon count, etc.) This technique may be applicable for wavelengths extending from the ultra-violet, through the visible, and into the mid-infrared portion of the spectrum.

The systems for sensing, analyzing, and/or monitoring components of a fluid (e.g., analytes of interest, etc.) may capture and process data iteratively or continuously on-the-fly as the targets are viewed for processing (e.g., as an entire reactant array or an entirety of a portion of the reactant array of a CSA is viewed for processing). The captured or obtained data (e.g., spectral data, etc.) may then be processed to accurately associate the captured or obtained data with data associated with a known component or condition. During a single analysis test of a target (e.g. a fluid analysis test or other suitable test), the target (e.g., a reactant array or a portion of the reactant array of a CSA or other suitable target) may be viewed for processing one or more times or continuously over a length of a test. By performing repetitive measurements over time, the changes to the target (e.g., changes of reflective spectra of some or all reactants of a reactant array of a CSA or other suitable changes) may be recorded and used to identify components and/or a condition of the target (e.g., one or more components of a fluid tested and/or more other suitable components or conditions).

In some cases, it may be desirable to analyze a target without scanning the target when analyzing and/or monitoring the target. For example, not being required to mechanically scan the target may facilitate creating a compact, low-cost system that may be handheld and/or accomplish measurements of the target in a short amount of time.

The systems for sensing, analyzing, and/or monitoring targets may enable a realization of Fourier transform hyperspectral imaging without a need for mechanical scanning of the targets (e.g., without relative movement of the reactants and the sensing or imaging components of the analysis system). An example Fourier transform hyperspectral imaging system is described in US Patent Application Publication No. 2021/0181022, filed on Feb. 18, 2021, and titled FOURIER-TRANSFORM HYPERSPECTRAL IMAGING SYSTEM, which is hereby incorporated by reference in its entirety for any and all purposes.

Although the use of Fourier transform hyperspectral imaging may be utilized in systems for sensing, analyzing, and/or monitoring for analytes in fluids, as the primary application discussed herein, the discussed designs or concepts may be utilized in other suitable applications. Example suitable applications include, but are not limited to, line scan-based agricultural crop growth monitoring, line scan-based analysis of antique objects (e.g., paintings, etc.), measuring spectrum of an arrayed object (e.g., arrayed objects served by a line scan camera in an industrial quality control production line, etc.), arrayed fluorescence excitation and collection, arrayed two or multiple photon excitation and up-conversion light collection applications, arrayed non-linear optics related light excitation and collection applications, applications in which hyperspectral cameras are utilized, and/or other suitable applications.

In the example application of sensing, analyzing, and/or monitoring for analytes in fluids as discussed herein, a principle of operation for enabling the realization of Fourier transform hyperspectral imaging without the need for mechanical scanning may be based on spatial low coherence interferometry in which each reactant may be considered as a diffused low coherence light source which is optically divided into two sub-sources to interfere with each other along a direction of light detector pixels of an image sensor, with each light detector pixel detecting the optically interfered light signal of a different relative optical path length difference so the interference pattern or interferogram is a Fourier transform of the spectral content of a reactant array of a CSA. By arranging different reactants of the reactant array along a direction orthogonal to a 2-dimensional (2D) light detector array image sensor, the 2D image sensor may be utilized to record the Fourier transform of the spectrum of all the reactants of the CSA (e.g., an interferogram representing the frequency domain of the light waves from the spectrum of the reactants of the CSA). As a result, an inverse (e.g., a reverse) Fourier transform of the interferogram may transform the interferogram from the spatial frequency domain to the spectral domain and reveal the original spectrum of light from all the reactants of the CSA, which may be accomplished without mechanically scanning the reactants.

The analysis system may include, among other components, an optical design that utilizes a combination of one or more lenses and one or more slits. In one example configuration, the optical design may utilize a cylinder lens in combination with an imaging lens (e.g., a spherical or aspheric lens) or a single toric lens (e.g., a lens with different optical power and focal length in two orientations perpendicular to each other) to guide light rays from a target area (e.g., the lines, rectangles, dots, etc. of the reactant) to one or more slits (e.g., two spatially separated slits) in an opaque structure followed by another imaging lens in combination with a cylinder lens or another single toric lens positioned between the opaque structure with the one or more slits and an image sensor. In some examples, light from the entire reactant array or an entire desired portion of the reactant array may simultaneously pass through the single slit or separated slits, including space between every two neighboring reactants that may act as “white” calibration space, such that an illumination component and/or a light collection component of the analysis system does not have to be adjusted relative to the reactant array (e.g., the CSA). When two slits are utilized in the opaque structure, the two slits may be sufficiently close to each other such that a wave front of light from the reactant may be sampled by the two slits from the same original source to optically interfere (e.g., in a manner similar to how the case of Young's double slit setup operates).

Young's double slit experiment includes applying a light beam from a single source (e.g., a target area) to two parallel, elongated slits spaced from one another and extending through an opaque surface or structure (e.g., member). To improve spatial coherence of waves received at the slits, an opaque surface or structure having a single slit may be placed in front of the opaque surface or structure with the two slits such that the single slit may act as a single light source for light received at the opaque surface or structure with the two slits. As long as there is spatial coherence of light received at the two slits, wave front division of the light received may result in the light that passes through the two slits interfering (e.g., wave fronts from the two slits may overlap with one another) to form an interferogram on a surface (e.g., a surface of a light or image sensor) that is at least a predetermined optical distance from the opaque surface or structure with the two slits. The interferogram may be a Fourier transform of the optical spectrum of the original light source.

In addition to or as an alternative to using two slits in the opaque structure, a prism may be utilized. When used, the prism may be configured to sample two portions of the original wave front of light from the reactants and bend the portions of the original wave front of light such that the portions overlap with each other. Further, in some cases, an optical amplitude division element such as a thin film beam splitter may be used to split the original light wave from the reactants and other free space optical element(s) can be used to combine and overlap the two optical waves with each other.

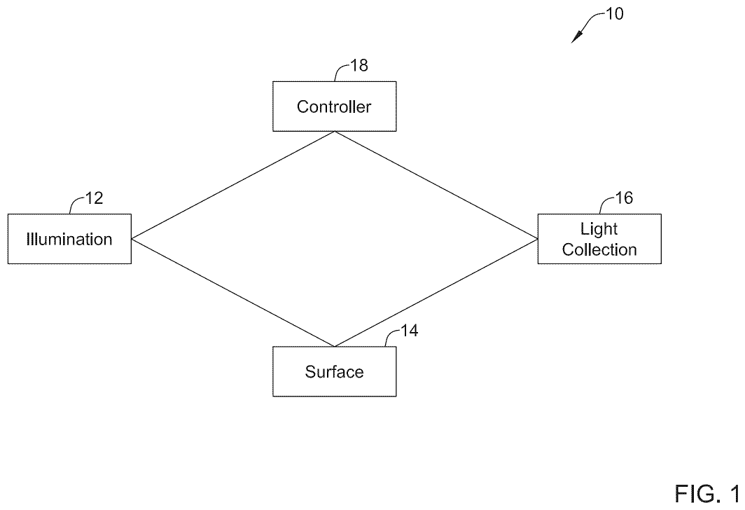

Turning to the Figures, FIG. 1 schematically depicts an illustrative configuration of an analysis system 10 (e.g., a Fourier transform hyperspectral fluid analysis system and/or other suitable analysis system) for determining a component and/or condition of or at a target. In some examples, the analysis system 10 may include, among other components, an illumination component 12 configured to illuminate a target area (e.g., in an example of a fluid analysis system, the target area may be or may include one or more analyte sensitive materials or reactants of a reactant array) on, supported by, or including a surface 14, a light collection component 16 configured to receive or collect light from the target area, and a controller 18 configured to be in communication with the illumination component 12 and/or the light collection component 16. The controller 18 may be configured to analyze or facilitate analyzing data related to light collected at the light collection component 16. In some instances, the illumination component 12 may be omitted.

When included in the analysis system 10, the illumination component 12 may include one or more light sources, an illumination lens system (e.g., one or more illumination lens subsystems), and/or other suitable components. The illumination component 12 may be configured to provide sufficient photons with a uniform spatial and spectral distribution spanning a wavelength range of interest for the target area.

The one or more light sources may be configured to provide any suitable wavelengths of light to the target area. In some examples, the one or more light sources may provide uniform spatial and spectral distributions of wavelengths of light spanning one or more ranges of, but not limited to, about 300 nanometers (nm) to about 1000 nm, a range of about 360 nm to about 900 nm, a range of about 350 nm to about 500 nm, a range of about 300 nm to about 600 nm, a range of about 400 nm to about 725 nm, a range of about 425 nm to about 725 nm, a range of 700 nm to about 1000 nm, a range of about 800 nm to about 1000 nm, and/or other suitable ranges of wavelengths of light. In one example, one or more light sources may provide wavelengths of light spanning a range of about 400 nm to about 725 nm.

The illumination component 12 may be configured to provide illumination light in two or more different discrete ranges of wavelengths of light. For example, the one or more light sources may provide light in a first range of wavelengths of light (e.g., about 300 nm to about 600 nm) and in a second range of wavelengths of light (e.g., about 800 nm to about 1000 nm). Providing illumination in two discrete ranges of wavelengths of light may be achieved by utilizing two or more light sources, through the use of filters, and/or in one or more other suitable manners. Having the ability to provide light in two or more discrete wavelength ranges may facilitate using the analysis system 10 for different applications that may require use of different wavelength ranges for optimal performance.

In some configurations, the one or more light sources may be configured to provide at least a uniform spatial and spectral distribution of broadband white light (e.g., continuous broadband white light) to the target area. In one example, the light source providing the uniform spatial and spectral distribution of broadband white light may provide light wavelengths spanning a range of about 360 nm to about 900 nm. In another example, the light source providing the uniform spatial and spectral distribution of broadband white light may provide light wavelengths spanning a range of about 400 nm to about 725 nm. Such configured light sources may have a desired (e.g., high) color rendering index (CRI), with a uniform distribution of photon wavelengths through the entire visible spectrum.

The one or more light sources may be any suitable type of light source. For example, the light source may be a light emitting diode (LED), an indium based blue LED with multiple phosphors added to a doping to create a combined LED and electro-luminescent semiconductor junction light emitting source, a black body radiation source, a tungsten lamp, a halogen lamp, and/or other suitable type of light source. In one example, the light source(s) may be a true color white LED configured to provide light wavelengths in a range of about 400 nm to about 725 nm, but other suitable configurations are contemplated. Utilizing a white LED rather than a black body radiation source (e.g., tungsten lamps, halogen lamps, etc.) may reduce inefficiencies of electron to photon conversion and allow the analysis system 10 to use less power (e.g., have a higher electron to photon conversion ratio) than when other types of light sources (e.g., tungsten lamps, halogen lamps, etc.) are used.

The light sources may be provided at any suitable angle and at any suitable location relative to the target area and/or the light collection component 16. For example, the light sources may be provided at angles in a range of about 0 degrees to about 90 degrees relative to the target area, at angles in a range of about 15 degrees to about 75 degrees relative to the target area, at angles in a range of about 30 degrees to about 60 degrees relative to the target area, at angles in a range of about 40 degrees to 50 degrees relative to the target area and/or at one or more other suitable angles. In one example, the light sources may be angled at 45 degrees relative to the target area, but other suitable configurations are contemplated. Providing light sources that project light onto the target area from an acute angle and from a location spaced laterally from a target area (e.g., a lighted area) on the surface 14 may facilitate providing dual overlapping ellipsoids that effectively form the target area (e.g., form a target area sized to cover one or more reactants or portions of the one or more reactants) to be analyzed while minimizing collection of spectral or specular reflection light and allowing for maximum diffuse light collection.

In some configurations, the illumination component 12 may include an illumination lens system configured to deliver and focus light from the light source on or to create a target area on the surface 14. In some examples, the target area on the surface 14 may cover or include one or more reactants on the surface 14, but other suitable target areas are contemplated. The illumination lens system may include any suitable components including, but not limited to, one or more lenses, one or more fiber optics, and/or one or more other suitable components.

In an example application of the analysis system 10, the analysis system 10 may be used in a fluid analysis test. When the analysis system 10 is used in a fluid analysis test to analyze a fluid, the target area may include one or more reactants (e.g., analyte sensitive materials) of a reactant array on, supported by, or of the surface 14 and the one or more reactants may be exposed to the fluid to be tested. In some examples, the one or more reactants may be exposed to fluid in any suitable manner including, but not limited to, by pumping fluid to or along the one or more reactants during a fluid test using the analysis system 10, exposing the one or more reactants to the fluid prior to being positioned in the analysis system 10, positioning the one or more reactants proximate an area of interest (e.g., a wound, etc.) prior to being positioned in the analysis system 10, and/or the one or more reactants may be exposed to fluid in one or more other suitable manners. Once the one or more reactants have been exposed to fluid for analysis of the fluid and light has been collected from the one or more reactants during a fluid analysis test, the controller 18 may analyze light collection data to identifying one or more components (e.g., analytes) of the fluid to which the one or more reactants were exposed.

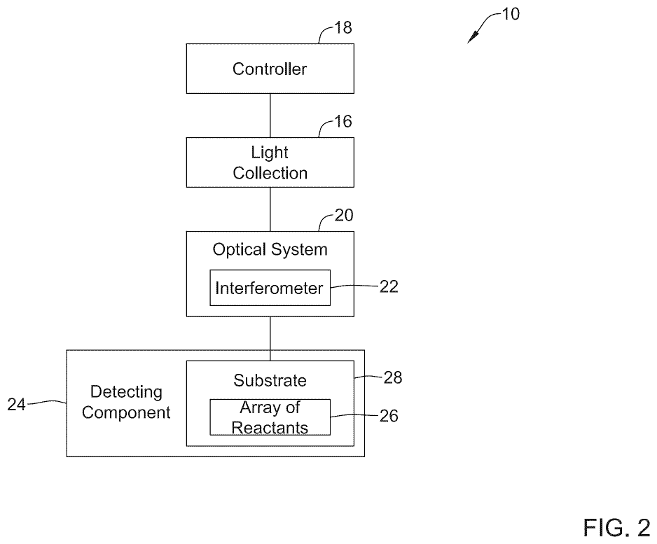

FIG. 2 schematically depicts a diagram of an illustrative configuration of the analysis system 10 configured for use in a fluid analysis test. The illustrative configuration of the analysis system 10 depicted in FIG. 2 may include, among other components, the light collection component 16, the controller 18, an optical system 20, and a detecting component 24 configured to sense an analyte, where the detecting component 24 may be adjustable or fixed relative to the light collection component 16 and/or the optical system 20. Although the analysis system 10 is depicted in FIG. 2 without the illumination component 12, the illumination component 12 may be included. Optionally, the analysis system 10 may include a housing configured to house one or more of the light collection component 16, the controller 18, the optical system 20, the detecting component 24, and/or other suitable components of the analysis system 10.

The detecting component 24 may include a reactant array 26 having the one or more reactants and a substrate 28 supporting the reactant array 26, where reactants of the reactant array 26 may be configured to react to exposure to a fluid tested in the fluid analysis test. In some examples, the substrate 28 may be or may include the surface 14 depicted in FIG. 1, but other configurations are contemplated.

The substrate 28 of the detecting component 24 may have any suitable configuration for supporting and/or receiving the reactant array 26 for exposure to a fluid (e.g., a fluid of interest) and/or for analysis of the reactant array 26 using the optical system and light collection component 16 of the system 10. For example, the substrate 28 may be sized to contain all of or a portion of the reactant array 26. In some examples, multiple substrates 28 may be utilized to contain all of or a portion of the reactant array 26. Additionally or alternatively, the substrate 28 and the reactant array 26 may be one in the same, such that the reactant array 26 or reactants thereof form the substrate 28.

The substrate 28 may take on, or may have a surface (e.g., the surface 14) that may be, any suitable shape including, but not limited to, an elongated shape, a rectangular shape, a square shape, a rounded shape, a spherical shape, a circular shape, a cylindrical shape, a disc shape, a triangle shape, a trapezoid shape, a prism shape, a lens shape, and/or other suitable shape. The substrate 28 may be or may include a surface of a container or cartridge or a component configured to be within a container or cartridge. In some instances, a cross-section of the substrate 28 may be symmetrical about a center line extending perpendicularly through a surface of the substrate 28 configured to support one or more reactants of the reactant array 26.

The substrate 28 may include and/or may be formed from any suitable material. Example suitable materials used for the substrate 28 of the detecting component 24 include, but are not limited to, polymers, optical polymers, optical glasses, plastic, rubber, glass, paper, filter material, filter paper, fabric, metal, aluminum, polypropylene, polytetrafluorethylenes, porous membranes, chromatography plates, acrylic (e.g., poly(methyl methacrylate) (PMMA)), polycarbonate (PC), polystyrene (PS), non-reactant materials, other suitable materials, and/or combinations thereof. Further, the material utilized for the substrate 28 may be a solid material, a woven material, a hydrophobic material, a gas permeable material, a gas impermeable material, other suitable materials, and/or combinations thereof.

In one example configuration of the substrate 28, the substrate 28 may be or may include a portion that is formed from a porous white plastic membrane (e.g., a material that does not react to analytes to be tested) that has a high diffuse reflectivity over an entire visible spectrum, at least a portion of the ultraviolet (UV) spectrum, and/or at least a portion of the infrared (IR) spectrum. When the substrate 28 is at least partially formed from a white plastic membrane that has a high diffuse reflectivity over at least an entire visible spectrum, the light collection component 16 of the analysis system 10 may be configured to collect a 100% white spectrum from the substrate 28, which may be used for fluid analysis purposes (e.g., to normalize results from the reactants).

In another example configuration of the substrate 28, the substrate 28 may be or may include a portion that is formed from a woven polypropylene material, which may result in a gas permeable, hydrophobic substrate 28. Although other pore sizes are contemplated, in the example configuration, the woven substrate may have an average pore size of or about 0.2 microns and a diameter of about 25 millimeters (mm). Additionally or alternatively, an example configuration of the substrate 28 may be formed from one or more other suitable hydrophobic, gas permeable materials.

In another example configuration of the substrate 28, the substrate 28 be or may include a portion that is formed from a transparent material (e.g., acrylic (e.g., poly(methyl methacrylate) (PMMA)), polycarbonate (PC), polystyrene (PS), etc.) configured to pass light from one surface of the transparent material through a second surface of the material. In some examples, the substrate 28 may be entirely transparent or include one or more transparent portions configured to illuminate the reactants of the reactant array 26 through the substrate 28 and/or collect light from the reactants of the reactant array 26 through the substrate 28. In some cases, the one or more transparent portions of the substrate 28 may extend between at least a first surface and a second surface of the substrate 28, where the first and second surfaces may be parallel or non-parallel with one another and the reactants are located on the first surface.

To increase fluid component detection rates by the reactants of the reactant array 26, the substrate 28 on which the reactant array 26 is applied and/or the reactants of the reactant array 26 may be textured (e.g., with grooves or surface topographical undulations, woven patterns, etc.) so as to increase an effective surface area of the reactants (e.g., the analyte sensitive material for detecting analytes). Additionally or alternatively, the reactants of the reactant array 26 may be formed from a textured material and the substrate 28 may or may not be omitted. Such texturing may be applied to the substrate 28 and/or the reactants of the reactant array 26 using any suitable technique including, but not limited to, via etching, thermoforming, pressure forming, molding, machining, weaving, three-dimensional printing, deposition, and/or other suitable techniques.

The reactants of the reactant array 26 may be formed from any suitable material. In some cases, the material of the reactants may be an optically responsive chemical material (e.g., a chemoresponsive material) that changes color in response to detecting one or more analytes (e.g., non-volatile and/or volatile compounds, gases, liquids, and/or other fluids) in a fluid to which the reactants are exposed. Example suitable materials for reactants include dyes from, but not limited to, the following classes: Lewis acid/base dyes (e.g., metal containing dyes), Brensted acidic or basic dyes (e.g., pH indicators), dyes with large permanent dipoles (e.g., solvatochromic dyes), redox responsive dyes (e.g., metal nanoparticle precursors), and/or other suitable classes of dyes. One example material for the reactants may be a silver nanoparticle material. Other suitable materials for the reactants are contemplated, including reactant material that is not a printed dye.

In some examples, the material of the reactants may include an analyte sensitive material that is reversible or semi-reversible. Reversible or semi-reversible analyte sensitive material may be utilized for reactants configured for repeat monitoring, such as for continuous or periodic sensing of target locations to detect analytes from the target locations. Although other configurations of reactant arrays 26 are contemplated, example reactant arrays 26 including analyte sensitive material that is reversible or semi-reversible are discussed in U.S. Pat. No. 6,368,558 filed on Mar. 21, 2000, and titled COLORIMETRIC ARTIFICIAL NOSE HAVING AN ARRAY OF DYES AND METHOD FOR ARTIFICIAL OLFACTION; U.S. Pat. No. 6,495,102 filed on Nov. 11, 2000, and titled COLORIMETRIC ARTIFICIAL NOSE HAVING AN ARRAY OF DYES AND METHOD FOR ARTIFICIAL OLFACTION; U.S. Pat. No. 7,261,857 filed on Oct. 24, 2002, and titled COLORIMETRIC ARTIFICIAL NOSE HAVING AN ARRAY OF DYES AND METHOD FOR ARTIFICIAL OLFACTION; U.S. Pat. No. 8,852,504 filed on Oct. 11, 2007, and titled APPARATUS AND METHOD FOR DETECTING AND IDENTIFYING MICROORGANISMS, all of which are hereby incorporated by reference in their entirety and for all purposes.

In some examples, the material of the reactants may include an analyte sensitive material that is irreversible. Irreversible analyte sensitive material may be utilized for reactants configured for single use monitoring or single use monitoring per analyte material of a fluid when the reactant array 26 is configured to monitor for a plurality of different analytes, but this is not required. Although other configurations of reactant arrays 26 are contemplated, example reactant arrays 26 including analyte sensing material that is irreversible are discussed in U.S. Pat. No. 9,880,137 filed on Sep. 2, 2009, and titled COLORIMETRIC SENSOR ARRAYS BASED ON NANOPOROUS PIGMENTS; U.S. Pat. No. 10,539,508 filed on Jun. 9, 2015, and titled PORTABLE DEVICE FOR COLORIMETRIC OR FLUOROMETRIC ANALYSIS AND METHOD OF CONDUCTING COLORIMETRIC OR FLUOROMETRIC ANALYSIS; Li, Zheng, et al., “Ultrasensitive Monitoring of Museum Airborne Pollutants Using a Silver Nanoparticle Sensor Array”, ACS sensors 5.9 (2020): 2783-2791; Li, Zheng, and Kenneth S. Suslick, “Chemically Induced Sintering of Nanoparticles”, Angewandte Chemie 131.40 (2019): 14331-14334; LaGasse, Maria K., et al., “Colorimetric sensor arrays: Development and application to art conservation”, Journal of the American Institute for Conservation 57.3 (2018): 127-140, all of which are hereby incorporated by reference in their entirety and for all purposes.

The reactants of the reactant array 26 may be applied to the substrate 28 in any suitable manner. In one example, the reactants may be applied to the substrate 28 by printing the reactants (e.g., the material of the reactants) on the substrate 28. When printed, any suitable printing techniques may be utilized including, but not limited to, pin transfer, inkjet, silkscreen, and/or other suitable application techniques.

The reactants may be applied to the substrate 28 randomly and/or to form one or more patterns. Example configurations of the reactants of the reactant array 26 applied to the substrate 28 include, but are not limited to, grid patterns of rows and columns, concentric rings, color matching of a color of printed dye material with a color of a substrate material prior to interactions with analyte, patterns that result in identifiable shapes when the analyte sensitive material reacts to a particular analyte, other suitable configurations, and/or combinations thereof.

A top surface and/or other suitable surface of the substrate 28 may be coated with a porous material to increase the surface area when reactants are applied to the substrate 28. In one example, the top surface (e.g., the surface 14) of the substrate 28 may be coated with a thin layer of porous material, such as a sol-gel and/or other suitable material.

The optical system 20 of the analysis system 10 may be entirely or at least partially positioned between the detecting component 24 and/or one or more other suitable target areas and the light collection component 16. The optical system 20 may include one or more lenses (e.g., a collection lens configuration) configured in the analysis system 10 to receive light from the target area and focus the light on a light or image sensor of the light collection component 16. Further, the optical system 20 may include one or more opaque members having one or more slits therein and/or an interferometer 22. In some examples, the one or more lenses and the interferometer 22 (e.g., where the interferometer may or may not include the opaque member with one or more slits) of the optical system 20 may be configured to form an interferogram on a light or image sensor of the light collection component 16 that is a Fourier transform of a spectrum of light received from the target area such that an inverse Fourier transform of the interferogram may reveal the spectrum of the light received from the target area.

The one or more lenses of the optical system 20 may be configured in the analysis system as being part of one or more sets of lenses. In some examples, the optical system 20 may include a first set of one or more lenses and a second set of one or more lenses, where the opaque member may be positioned between the first set of one or more lenses and the second set of one or more lenses. In one example using the fluid analysis application, the first set of one or more lenses may be configured to form an image of the reactant array 26 or a portion thereof at the opaque member and the second set of one or more lenses may be configured to form an interferogram on a surface (e.g., a surface of a light or image sensor of the light collection component), but other suitable configurations are contemplated.

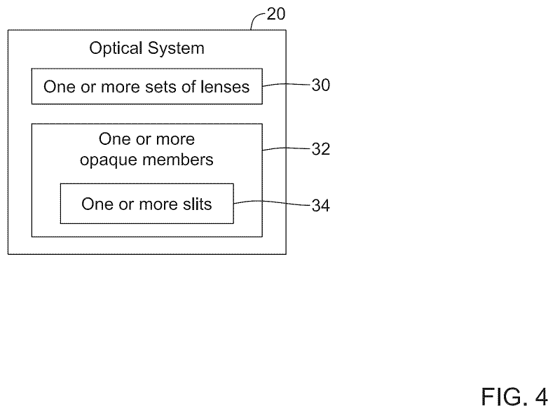

The interferometer 22 may include the opaque member 32 (e.g., as depicted in FIGS. 4-16 or otherwise). For example, when the opaque member 32 includes two or more slits configured to act as separate sources of light that are able to create an interference pattern (e.g., the interferogram) that can be sensed, measured, and analyzed with the light collection component 16 and/or the controller 18, the opaque member 32 may be considered to be or to be part of the interferometer 22. Alternatively or additionally, the interferometer 22 may take on one or more other suitable configurations configured to receive light from the target area (e.g., after the light has passed through a slit in the opaque member or light that has not passed through the opaque member), divide the received light into two or more beams, and result in the two or more beams interacting to create an interferogram.

The light collection component 16 (e.g., diffuse reflection capture optics, etc.) may be configured to collect and/or measure levels of or changes in wavelengths of light collected from the surface 14 (e.g., measure photons by wavelengths of light from reactants of the reactant array 26) and may include one or more light collectors configured to receive light from the optical system 20 and/or may include one or more other suitable components. The light collection component 16 may be positioned at any suitable location relative to the detecting component 24. In some examples, the light collection component 16 may be configured to collect light from a same side of the detecting component 24 from which the illumination component 12, when included, illuminates the detecting component 24, from a different side of the detecting component 24 than from which the illumination component 12 illuminates the detecting component 24, directly from the reactant of the reactant array 26, indirectly through a transparent substrate 28 of the detecting component 24, and/or from one or more other suitable locations and/or in one or more other suitable manners.

The light collection component 16 may include one or more fiber optics (e.g., one or more optical fibers or a fiber array or waveguide array) configured (e.g., tuned and positioned) to receive light from or focus light from one or more reactants of the reactant array 26, where the light received at the fiber optics may have traveled through at least part of or an entirety of the optical system 20. The one or more fiber optics may be or may include single mode and/or multimode fiber optics, as desired. The one or more fiber optics may have a first end configured to receive or collect light from the target area and a second end in optical communication with the light collector.

The light collection component 16 may include one or more light collectors of any suitable type. Example suitable types of light collectors may include, but are not limited to, a light sensor, an image sensor, an n-dimensional sensory array (e.g., where “n” equals 1, 2, etc.), a linear 2D light detector array image sensor, light detector array image sensor may include, a spectrometer, a charge-coupled device (CCD) image sensor, complementary metal-oxide semiconductor (CMOS) image sensor, contact image sensor (CIS), color contact image sensor (CCIS), a camera, other suitable light collectors, and/or combinations of light collectors. In one example, the light collector may include a spectrometer configured to measure photons collected from (e.g., reflected, transmitted, and/or otherwise received from) the target area. Utilizing a spectrometer may facilitate sensing wavelengths of light with high resolution in the nanometer range and may provide a continuous set of data over the wavelength range, which allows for a sensitive analysis of the data to identify components of a fluid to which the reactant array 26 was exposed relative to when other light collectors are used. In another example, the light collector may include a 2D pixel array image sensor configured to record multiple spatial interferograms in a pixel array direction of an interferogram representing a Fourier transform of the reactant array 26, which may provide sufficient sensitivity, while being compact and cost-effective.

In some examples, a pixel density and image sensor size of the light or image sensor may be selected based on optical parameters of lenses and/or other components of the analysis system 10 such that the number of pixels is sufficient and dense enough to capture a full range of interferograms that cover a wavelength range of a full visible spectrum as well as some of the near infrared spectrum. The pixel density of the light or image sensor may ensure a highest spatial frequency is not limited by the Nyquist frequency of the light or image sensor and at the same time the reverse Fourier transform can produce a spectrum of the reactant array 26 with a resolution in a desired range (e.g., such as a nanometer range).

The controller 18 may be coupled to one or more other electronic components of the analysis system 10. For example, the controller 18 may be communicatively coupled with one or more of the illumination component, when included, the light collection component 16, the optical system 20, and/or one or more other suitable components of the analysis system 10 and/or remote components (e.g., servers, mobile devices, etc.) that may or may not be part of the analysis system 10. In some examples, the controller 18 may be configured to receive an indication to initiate a fluid analysis test (e.g., from a user via a user interface or in communication with the controller 18) and send coordinated control signals to one or more electronic components of the analysis system 10.

The controller 18 may be configured to identify or may facilitate identifying a component of fluid in contact with the detecting component 24 (e.g., including the surface 14) and/or a condition of a target area based on measured (e.g., sensed and/or calculated) levels of light (e.g., interferograms) or changes in light sensed or collected from the detecting component 24 with the light collection component 16. In some examples, the controller 18 may be configured to identify a component of fluid in contact with the detecting component 24 and/or a condition of the target area based on one or more of a timing of levels of the wavelength of light from the target area and an absolute change between a level of a wavelength of light collected from the target area at a time of or prior to an application of the fluid to the detecting component 24 and at a predetermined time after initially applying the fluid to the detecting component 24, and levels of light from the target area relative to predetermined or expected levels of light from the target area. The controller 18 may be configured to identify the component of the fluid in contact with the detecting component 24 or a condition at the target area based on light from the target area that is received at the light collection component 16 in one or more additional or alternative manners.

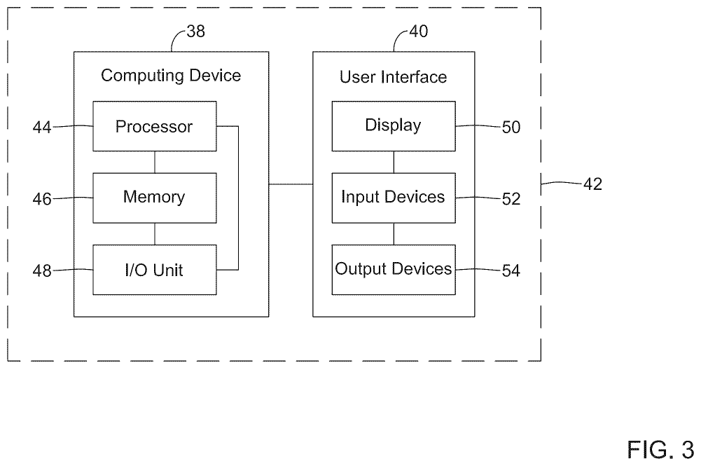

The controller 18 and/or other components of the analysis system 10 may be or may include one or more computing devices including or coupled with one or more user interfaces. FIG. 3 depicts a schematic diagram of an illustrative computing device 38 and a user interface 40, where the computing device 38 and/or the user interface 40 may be entirely or partially housed in one or more housings 42 (e.g., a housing which may or may not house other components of the analysis system 10). The housing 42 may be an optional component, as represented by the broken lines defining the housing 42 depicted in FIG. 3. Although various components are depicted as being included in the computing device 38 and the user interface 40, one more of the depicted components may be omitted and/or one or more additional or alternative components may be utilized.

The computing device 38 may be any suitable computing device configured to process data of or for the analysis system 10 and may be configured to facilitate operation of the analysis system 10. The computing device 38, in some cases, may be configured to control operation of the analysis system 10 by establishing and/or outputting control signals to the light collection component 16 and/or other electronic components of the analysis system 10 to run a test on target areas or fluid passing by or at (e.g., located at, trapped at, contained by, in, from, etc.) the target area and/or monitor results of a test. In some examples, the computing device 38 may be part of the controller 18 and may communicate with other components over a wired or wireless connection, but other suitable configurations are contemplated. When the computing device 38, or at least a part of the computing device 38, is a component separate from a structure of the controller 18, the computing device 38 may communicate with electronic components of the analysis system 10 over one or more wired or wireless connections or networks (e.g., LANs and/or WANs). In some cases, the computing device 38 may communicate with a remote server or other suitable computing device.

The illustrative computing device 38 may include, among other suitable components, one or more processors 44, memory 46, and/or one or more I/O units 48. Example other suitable components of the computing device 38 that are not specifically depicted in FIG. 3 may include, but are not limited to, communication components, a touch screen, selectable buttons, and/or other suitable components of a computing device. As discussed, one or more components of the computing device 38 may be separate from the controller 18 and/or incorporated into the components of the controller 18.

The processor 44 of the computing device 38 may include a single processor or more than one processor working individually or with one another. The processor 44 may be configured to receive and execute instructions, including instructions that may be loaded into the memory 46 and/or other suitable memory. Example components of the processor 44 may include, but are not limited to, central processing units, microprocessors, microcontrollers, multi-core processors, graphical processing units, digital signal processors, application specific integrated circuits (ASICs), artificial intelligence accelerators, field programmable gate arrays (FPGAs), discrete circuitry, and/or other suitable types of data processing devices.

The memory 46 of the computing device 38 may include a single memory component or more than one memory component each working individually or with one another. Example types of memory 46 may include random access memory (RAM), EEPROM, flash, suitable volatile storage devices, suitable non-volatile storage devices, persistent memory (e.g., read only memory (ROM), hard drive, flash memory, optical disc memory, and/or other suitable persistent memory) and/or other suitable types of memory. The memory 46 may be or may include a non-transitory computer readable medium. The memory 46 may include instructions stored in a transitory state and/or a non-transitory state on a computer readable medium that may be executable by the processor 44 to cause the processor 44 to perform one or more of the methods and/or techniques described herein. Further, in some cases, the memory 46 and/or other suitable memory may store data received from the light collection component 16 and/or other components of or in communication with the analysis system 10.

The I/O units 48 of the computing device 38 may include a single I/O component or more than one I/O component each working individually or with one another. Example I/O units 48 may be or may include any suitable types of communication hardware and/or software including, but not limited to, communication components or ports configured to communicate with electronic components of the analysis system 10 and/or with other suitable computing devices or systems. Example types of I/O units 48 may include, but are not limited to, wired communication components (e.g., HDMI components, Ethernet components, VGA components, serial communication components, parallel communication components, component video ports, S-video components, composite audio/video components, DVI components, USB components, optical communication components, and/or other suitable wired communication components), wireless communication components (e.g., radio frequency (RF) components, Low-Energy BLUETOOTH protocol components, BLUETOOH protocol components, Near-Field Communication (NFC) protocol components, WI-FI protocol components, optical communication components, ZIGBEE protocol components, and/or other suitable wireless communication components), and/or other suitable I/O units 48.

The user interface 40 may be configured to communicate with the computing device 38 via one or more wired or wireless connections. The user interface 40 may include one or more display devices 50, one or more input devices 52, one or more output devices 54, and/or one or more other suitable features. In some examples, the user interface 40 may be part of or may include the computing device 38.

The display 50 may be any suitable display. Example suitable displays include, but are not limited to, touch screen displays, non-touch screen displays, liquid crystal display (LCD) screens, light emitting diode (LED) displays, head mounted displays, virtual reality displays, augmented reality displays, and/or other suitable display types.

The input device(s) 52 may be and/or may include any suitable components and/or features for receiving user input via the user interface 40. Example input device(s) 52 may include, but are not limited to, touch screens, keypads, mice, touch pads, microphones, selectable buttons, selectable knobs, optical inputs, cameras, gesture sensors, eye trackers, voice recognition controls (e.g., microphones coupled to appropriate natural language processing components) and/or other suitable input devices. In one example, the input devices 52 may include a touch screen that allows for setting set points, initiating a fluid or target area analysis test, adjusting between screens (e.g., a testing screen, a data analysis screen, a results screen, etc.), and/or allows for taking one or more other suitable actions.

The output device(s) 54 may be and/or may include any suitable components and/or features for providing information and/or data to users and/or other computing components. Example output device(s) 54 include, but are not limited to, displays, speakers, vibration systems, tactile feedback systems, optical outputs, and/or other suitable output devices.

FIG. 4 depicts a schematic diagram of an illustrative configuration of the optical system 20. As discussed, the optical system 20 may include one or more sets of lenses 30 and one or more opaque members 32 including or defining one or more slits 34. In one example configuration, the optical system 20 may include a first set of lenses 30, a second set of lenses 30, and an opaque member 32 having one or more slits 34 and positioned between the first set of lenses 30 and the second set of lenses 30, where the first set of lenses 30 may be configured to provide light from the target area (e.g., provide an image or light beams or rays from the target area, such as an image of an array of reactants) to the opaque member 32 (e.g., to an intermediate image plane at the one or more slits 34 in the opaque member 32) and the second set of lenses 30 may be configured to form or otherwise focus an interferogram from light passing through the one or more slits on a surface (e.g., on a final image plane of a surface of the light collection component 16 and/or other suitable surface). Although not required, a housing (e.g., the housing 42 and/or other suitable housing) may be configured to house all of or at least a portion of the one or more sets of lenses 30 (e.g., the first set of lenses 30 and the second set of lenses 30) and the one or more opaque members 32.

The lenses of the one or more sets of lenses 30 may include any suitable type(s) of lenses configured (e.g., tuned and/or positioned) to receive, collect, and/or focus light from the target area (e.g., from one or more reactants of the reactant array 26) and direct the light to a surface (e.g., a surface of the one or more opaque members, a surface of a waveguide of the light collection component 16, a surface of a light or image sensor of the light collection component 16, and/or other suitable surface). Example suitable types of lenses of the one or more sets of lenses 30 may include, but are not limited to, imaging lenses, focusing lenses, spherical lenses, aspheric lenses, cylinder lenses, toric lenses, adjustable lenses, tunable liquid lenses, and/or one or more other suitable types of lenses. An example focusing lens may be a cylinder lens configured to collect light from a reactant array and/or direct light to a waveguide and/or a light or image sensor of the light collection component 16. Example imaging lenses may be spherical lenses and/or aspheric lenses configured to optically collimate and project light or an image onto the one or more slits 34 of the opaque member 32 and/or collect light passing through the one or more slits 34 of the opaque member 32 and collimate and project the collected light to the focusing lens.

The one or more sets of lenses may include any suitable configuration of lenses and may include sets of lenses 30 with a single lens, sets of lenses 30 with two or more lenses that may be similar to or different than one another, sets of lenses with one or more fixed location lenses, sets of lenses with one or more adjustable location lenses, sets of lenses with one or more adjustable focal points, and/or sets of lenses with other suitable lenses. Examples configurations of the one or more lenses of a set of lenses 30 may include, but are not limited to, a single lens such as a focusing lens (e.g., a cylinder lens and/or other suitable focusing lens) or an imaging lens (e.g., a spherical lens and/or other suitable imaging lens), a combination of a focusing lens and an imaging lens, a toric lens designed or configured to perform the functions of the focusing lens and the imaging lens, a tunable liquid lens, and/or other suitable configurations of the one or more lenses of a set of lenses 30. Configurations of the one or more lenses of the one or more sets of lenses 30 are discussed in greater detail herein.

The one or more opaque members 32 may have any suitable configuration configured to prevent light from the target area from passing between a first side of the opaque member(s) 32 (e.g., a side facing a direction of the target area) and a second side of the opaque member(s) 32 (e.g., a side facing a direction of the light collection component 16), except at the one or more slits. In examples when the one or more slits 34 include a single slit 34 configured to receive light from target area, the one or more slit(s) 34 may create a single source of light from the target area for an interferometer 22 positioned between the one or more opaque member 32 with the single slit 34 and the light collection component 16. In examples when the one or more slits 34 in the opaque member 32, include two or more slits 34 configured to create or form the interferometer 22 with the opaque member 32, the two or more slits 34 may be spaced from each other by any suitable distance that is similar to a wavelength or a few times or a few ten times of the wavelength of the light received from the target area and that results in a combined width of the all of the slits 34 and the spacing therebetween being less than a width of the light (e.g., light beams or rays) received from the target area.

The one or more slits 34, individually or in combination, may have or form any suitable diameter or width and height that is less than a diameter or width and a height of the beam of light from the target area (e.g., provided via a set of lenses 30). In some examples, the slits 34 and/or spaces between two slits 34 may have the same or different diameters or heights and widths depending on optical parameters of the optical system 20 and on a pixel count and/or density of a light or image sensor of the light collection component 16.

The one or more slits 34 may have any suitable configuration. In some examples, the slits 34 may be elongated, circular, and/or one or more other suitable shapes or configurations. In some examples, the slit 34 may be an opening in the opaque member 32, may be defined by an optical fiber array or waveguide array extending through the opaque member 32, and/or may be defined by other suitable objects or materials through which light travels and that extend through the opaque member 32.

When two or more slits 34 are utilized, the two or more slits 34 may be parallel and spaced with respect to one another and/or oriented in one or more other suitable manners such that light beams or rays passing through the two or more slits 34 may interfere with one another to form an interferogram on a surface. In some examples, the configuration of slits 34 may be similar to the configuration of slits utilized in a Young's double slit experiment to create two wave fronts from a single source of light, where the two wave fronts interfere with one another to form an interferogram on a surface.

The one or more slits 34 may be transverse to an elongated direction of an area of interest at the target area. For example, when the target area includes elongated reactants, a length direction of the individual slits 34 may be orthogonal to the elongated reactants. Alternatively or additionally, when the target area includes a linear array of reactants, a length direction of the individual slits 34 may be orthogonal to the elongated direction of each reactant and parallel to the linear direction of the linear array of reactants.

As discussed, one or more opaque members 32 may be utilized in the optical system 20. When two or more opaque members 32 are utilized in the optical system 20, two or more opaque members 32 may be at a same axial location between the target area and the light collection component 16 and/or two or more opaque members 32 may be spaced axially from one another between the target area and the light collection component 16. In one example configuration of using two opaque members 32, a first opaque member 32 may be located at a first axial location and may include a single slit 34 and a second opaque member 32 may be located at a second axial location spaced toward the light collection component 16 from the first opaque member 32 and may include two slits 34, where the slit 34 of the first opaque member 32 and the light passing therethrough may act as a single light source for the two slits 34 of the second opaque member 32. Use of an opaque member 32 with a single slit 34 in front of an opaque member 32 with two slits 34 may facilitate improving a spatial coherence of light received at the opaque member 32 with two slits 34 relative to when the opaque member 32 with the single slit 34 is not utilized. Other suitable configurations of the one or more opaque members 32 are contemplated, as discussed herein or otherwise.

Although not depicted in FIG. 4, the optical system 20 may include one or more mirrors. In terms of the overall configuration of the optical system 20, a design of the optical system 20 that utilizes mirrors may result in a more compact analysis system 10 than when mirrors are not included as mirrors may facilitate folding a light path such that a same optical air space may be used for a light beam to pass through more than once.

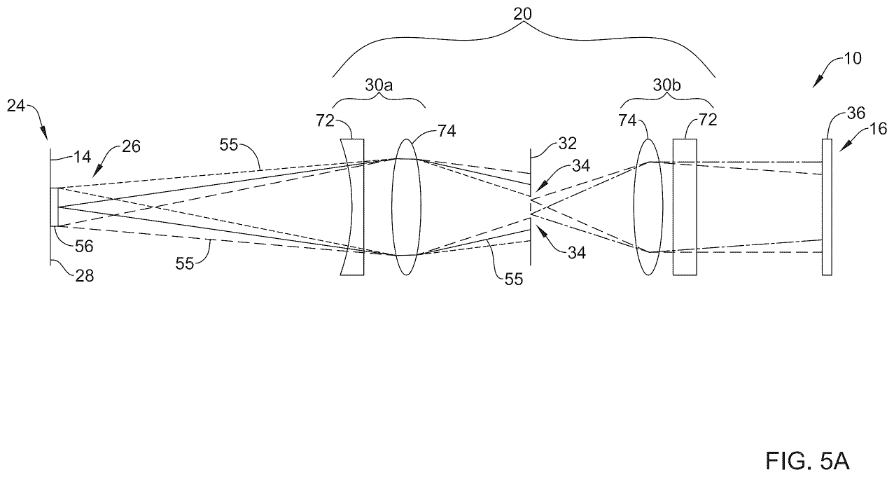

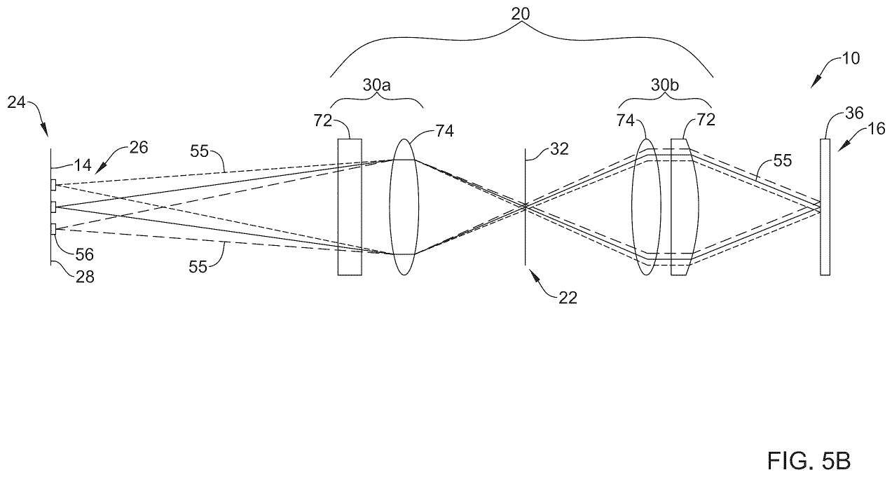

FIGS. 5A and 5B schematically depict a side view and a top view (e.g., views along orthogonal planes), respectively, of an illustrative configuration of the analysis system 10 that may enable Fourier transform hyperspectral imaging without a need for mechanical scanning of a target area. As depicted in FIGS. 5A and 5B, the target area may include the detecting component 24 with reactants 56 of the reactant array 26 supported by the substrate 28. Although no illumination component is depicted in the analysis system 10 depicted in FIGS. 5A and 5B, one or more illumination components may be utilized in the analysis system 10, as desired.

In FIGS. 5A and 5B, light beams or rays 55 may travel from the reactants 56 of the reactant array 26 to a light collector (e.g., a light or image sensor 36) of the light collection component 16. The different lines (e.g., different solid and broken lines) in FIG. 5A schematically represent light beams or rays 55 from different portions of the reactant 56 and the different lines in FIG. 5B schematically represent light beams or rays 55 from different reactants 56 of the reactant array 26.

The optical system 20 depicted in FIGS. 5A and 5B may include a first set of lenses 30a, a second set of lenses 30b, and an opaque member 32 positioned between the first set of lenses 30a and the second set of lenses 30b. The first set of lenses 30a may have any suitable configuration for providing light from the target area to the opaque member 32 and the second set of lenses 30b may have any suitable configuration for providing light from the opaque member 32 to the light collection component 16.

The first set of lenses 30a may be configured to focus or form an intermediate image of the reactant array 26 or a portion thereof at the opaque member 32 using a single lens (e.g., a single imaging lens 74) or multiple lenses. In the example of FIGS. 5A and 5B, the first set of lenses 30a may include a focusing lens 72 and an imaging lens 74, where the focusing lens 72 may be positioned between the reactant array 26 and the imaging lens 74 and the imaging lens 74 may be positioned between the focusing lens 72 and the opaque member 32.

As depicted in FIG. 5B, the combination of the focusing lens 72 and the imaging lens 74 of the first set of lenses 30, may focus the light beam or rays 55 from or form an image of the reactant array 26 at the opaque member 32 (e.g., at an intermediate plane at the opaque member 32). As can be seen from the side view of FIG. 5A, the light beam or rays 55 from the reactant array 26 may not be fully focused in such a way that all light (e.g., source points) along a length of each reactant 56 of the reactant array 26 will have light beams or rays 55 passing through the two slits 34. Similarly, light from outside of a length of each reactant 56 will not pass through the two slits 34.

Further in one illustrative configuration of the optical system 20, a second opaque member having a single slit 34 may be utilized between the opaque member 32 and the reactant array 26. When included, the second opaque member 32 with the single slit 34 may be positioned to right side of the first set of lenses 30a in front of the first opaque member 32, where light passing through the single slit 34 may act as light from a single source. Such a configuration of the second opaque member 32 may facilitate ensuring spatial coherence of light from the reactant array 26 and reducing a size of the analysis system 10 by selecting a portion of the light from the reactant array 26 (e.g., the portion of the light from the reactant array 26 that passes through the single slit 34) for analysis with the interferometer 22 and the light collection component 16.

The focusing lens 72 of the first set of lenses 30a may be any suitable focusing lens. For example, the focusing lens 72 between the reactant array 26 and the opaque member 32 may be one or more of negative cylinder lenses, one or more positive cylinder lenses, one or more prisms, one or more mirrors, and/or other suitable focusing lens 72 configured to bend light rays differently in two directions (e.g., two orthogonal directions and/or other suitable directions) to facilitate passing light from the entire reactant array 26 or a portion thereof to the slits 34 of the opaque member 32. In one example, the focusing lens 72 of the first set of lenses 30a may be a negative focusing lens, but other suitable configurations are contemplated.

The imaging lens 74 of the first set of lenses 30a may be any suitable imaging lens. For example, the imaging lens 74 between the reactant array 26 and the opaque member 32 may be an achromatic lens, a spherical lens, an aspheric lens, and/or other suitable type of imaging lens configured to form an image of the reactants 56 on or at the opaque member 32 (e.g., an intermediate image plane). In some examples, a focal plane of the imaging lens 74 of the first set of lenses 30a may be at the opaque member 32 (e.g., at the two slits 34 of the opaque member 32) and the imaging lens 74 may optically relay and propagate the light from the reactant array to the two slits 34 of the opaque member 32. In some examples, an image quality of the image produced by the imaging lens 74 to the left of the opaque member 32 should be sufficiently high such that each reactant 56 of the reactant array 26 may be separately identified from its adjacent reactants 56. When light beams or rays emerge from the two slits 34 of the opaque member 32, the emerging light beams or rays have wave fronts that optically interfere with one another to form an interferogram at the light or image sensor 36.

The function of the focusing lens 72 and the imaging lens 74 of the first set of lenses 30a may be replaced with two focusing lenses 72 (e.g., two cylinder lenses) in some cases, including, for example, when focusing powers along two perpendicular meridian planes are different such that a relatively sharply focused image of the reactants 56 along an elongated direction of the reactant array 26 may be formed at the opaque member 32 (e.g., as depicted in FIG. 5B) and along the other meridian direction, light beams or rays 55 from along each reactant length direction can travel to and pass through the two slits 34 in the opaque member 32 (e.g., as depicted in FIG. 5A). A similar function may be achieved by one lens having different orthogonal cylinder focusing powers (e.g., a toric lens and/or other suitable type of lens).

The second set of lenses 30b may be configured to focus or form an interferogram from light beams passing through the two slits 34 of the opaque member 32 on a final image plane at a surface (e.g., a surface of or in communication with a light or image sensor 36 of the light collection component 16) using a single lens (e.g., a single imaging lens 74) or multiple lenses. In the example of FIGS. 5A and 5B, the second set of lenses 30b may include a focusing lens 72 and an imaging lens 74 (both may be different from those in the first set of lenses 30a although the numerals used are the same), where the imaging lens 74 may be positioned between the opaque member 32 and the focusing lens 72 and the focusing lens 72 may be positioned between the imaging lens 74 and the light collection component 16. In some examples, the second set of lenses 30b may be configured to reduce a physical distance between the surface receiving the interferogram and the opaque member 32 with the double slits 34, while maintaining a sufficient optical distance for light to travel between the opaque member 32 and the surface to ensure an interferogram at the surface is an accurate Fourier transform of an optical spectrum from the reactant array 26.

The imaging lens 74 of the second set of lenses 30b may be any suitable imaging lens 74. In one example, the imaging lens 74 between the opaque member 32 and the light or image sensor 36 may be an achromatic lens, a spherical lens, an aspheric lens, and/or other suitable type of imaging lens 74. A forward focal plane of the imaging lens 74 of the second set of lenses 30b may be at opaque member 32 (e.g., at the two slits 34 of the opaque member 32) and the imaging lens 74 may collimate and propagate the light from the two slits 34 to the focusing lens 72.

The focusing lens 72 of the second set of lenses 30b may be any suitable focusing lens. For example, the focusing lens 72 between the opaque member 32 and the light or image sensor 36 may be one or more negative cylinder lenses, one or more positive cylinder lenses, one or more prisms, one or more mirrors, and/or other suitable focusing lens 72 configured to bend light rays differently in two directions (e.g., two orthogonal directions and/or other suitable directions) to facilitate optically forming an interferogram (e.g., an image of the reactant array or a portion thereof) on a surface (on a final image plane) of the light or image sensor 36. In one example, the focusing lens 72 may be a positive cylinder lens, but other suitable configurations are contemplated. In some configurations, the focusing lens 72 may have focusing power in only a single plane (e.g., the plane depicted in FIG. 5B), but other configurations are contemplated.

In some configurations, the imaging lens 74 and/or the focusing lens 72 of the second set of lenses 30b may be omitted and/or replaced with one or more other suitable lenses as long as (e.g., at least from the perspective depicted in FIG. 5A) a front focal plane of an effective lens with a focusing power on this plane is located at the slits 34 of the opaque member 32 and a resulting image from the reactant array 26 (e.g., the interferogram) may be formed on the surface of the light or image sensor 36 (e.g., the final image plane). Similar to the lenses of the first set of lenses 30a, the focusing lens 72 to the right of the opaque member 32 may be either a positive cylinder lens or a negative cylinder lens and the imaging lens 74 may be replaced with two cylinder lenses as long as the focusing powers along two orthogonal meridian planes are different such that a relatively sharply focused image of the reactant array 26 along a linear array direction of the reactant array 26 can be formed at the surface of the light or image sensor 36 in a first meridian direction (e.g., as depicted in FIG. 5B) and along a second meridian direction, light beams or rays 55 along the length direction of each reactant 56 can be collimated to enable the formation of spatial interferograms along that direction (e.g., as depicted in FIG. 5A). A similar function may be achieved by one lens having different orthogonal cylinder focusing powers (e.g., a toric lens and/or other suitable type of lens).

As discussed herein, the light or image sensor 36 of the light collection component 16 may be any suitable type of sensor. In some examples, the light or image sensor 36 may be a 2D pixel array image sensor (e.g., a 2D pixel array monochrome silicon-based image sensor, etc.) or other suitable light or image sensor having a surface (e.g., a sensing or detection surface) arranged at a final image plane of the optical system 20 (e.g., of the second set of lenses 30b). In such an example, interferograms may be recorded in a first direction on the sensing plane (e.g., in the plane of FIG. 5A) and light from different reactants 56 of the reactant array 26 may be optically separated from each other as an optically magnified or de-magnified image of the different reactants in a second orthogonal direction on the sensing plane (e.g., in the plane of FIG. 5B).

In some configurations the first set of lenses 30a between the target area and the opaque member 32 may be adjustable to provide adaptation to targets of different distances from the opaque member 32 and/or of different dimensions. An analysis system 10 utilizing a zoom function may be configured to facilitate accurately sensing changes in the reactant array 26 and/or may facilitate use of the analysis system 10 in other applications including, but not limited to, line scanning agricultural crop growth, line scanning forest for health/disease conditions, line scanning forests for fire monitoring, analyzing antique objects, monitoring quality in an industrial production line, and/or facilitating use of the analysis system 10 in one or more other suitable applications.

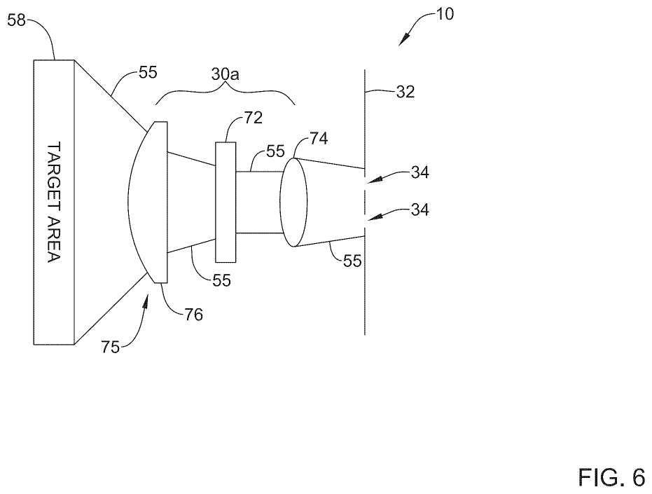

FIGS. 6-8 depict example lens configurations that may be adjustable and may be usable with, may include, and/or may replace the focusing lens 72 and/or the imaging lens 74 of the first set of lenses 30a. The adjustable lens and/or zoom functions discussed herein may be adjusted in response to control signals from the controller 18 and/or manually adjusted. Further, the adjustments of the zoom systems may be performed automatically by the analysis system 10 to obtain the best data possible based on an open loop or closed loop control configuration.

FIG. 6 depicts a portion of the analysis system 10 including an illustrative configuration of the first set of lenses 30a with an adjustable lens of a zoom system 75. As depicted in FIG. 6, the first set of lenses 30a may include the focusing lens 72 between the opaque member 32 and a target area 58 (e.g., the reactant array 26 and/or other suitable target area) and the imaging lens 74 between the focusing lens 72 and the opaque member 32. Further, the first set of lenses 30a may include a focus adjustable macro lens 76 of the zoom system 75 positioned between the focusing lens 72 and the target area 58 and configured to optically relay light from an entirety of or a portion of the target area 58 that is large relative to and/or close to the focusing lens 72, the imaging lens 74, and/or the opaque member 32. In such instances, the adjustable macro lens 76 may be configured to be adjusted to capture light from a large area relative to a diameter of the focusing lens 72 and/or the imaging lens 74. Utilization of the adjustable macro lens 76 may facilitate reducing a distance between the opaque member 32 and the target area 58, such that the analysis system 10 may take on a compact form (e.g., a handheld form). In some cases, the zoom system 75 with the adjustable macro lens 76 may be implemented in the first set of lenses 30a by adding an extension to a configuration including the focusing lens 72 and the imaging lens 74 or a lens configuration with an equivalent function.

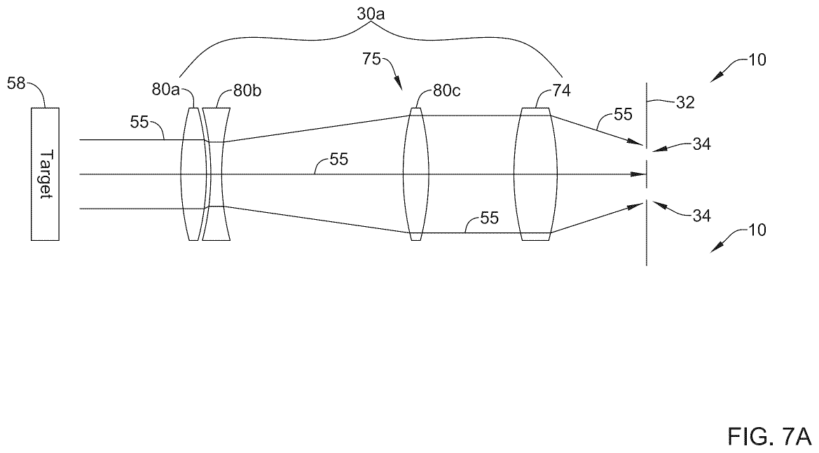

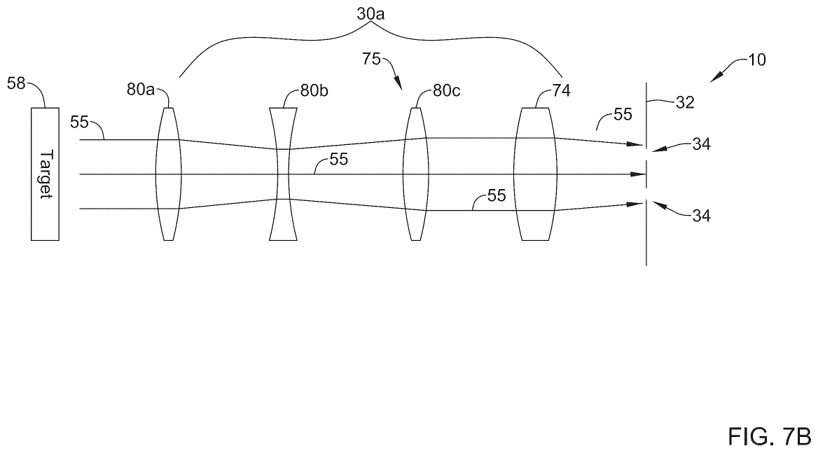

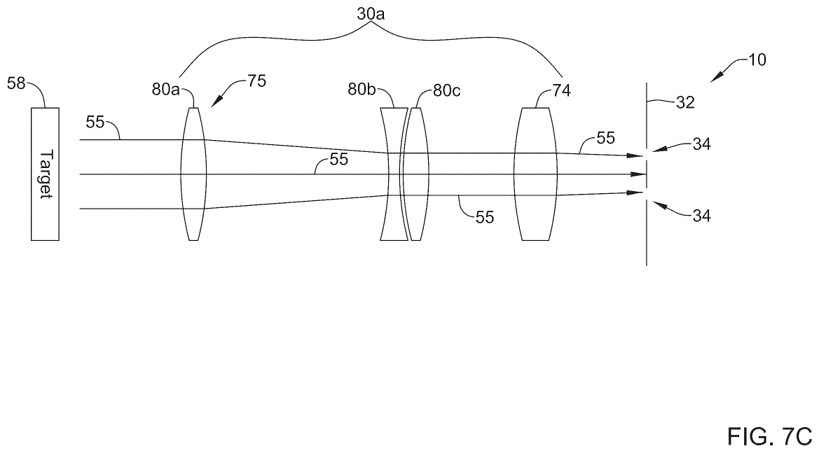

FIGS. 7A-7C depict schematic diagrams of the analysis system 10 including the first set of lenses 30a with an illustrative configuration of the zoom system 75 including an adjustable focal (e.g., afocal) zoom configuration. As depicted in FIGS. 7A-7C, zoom system 75 with the afocal zoom configuration may be utilized with a fixed focal length lens, such as the imaging lens 74. The first set of lenses 30a between the target area 58 and the opaque member 32 with the afocal zoom configuration of the zoom system 75 between the imaging lens 74 and the target area 58 may be configured to optically relay light from targets at the target area 58 to the opaque member 32 (e.g., at an intermediate imaging plane of the imaging lens 74), where the targets at the target area 58 may be different distances from the first set of lenses 30a or the opaque member 32 and/or of different dimensions from one another.

Depending on the configuration of the targets of the target area 58, the focusing lens 72 may be added to the first set of lenses depicted in FIGS. 7A-7C between the afocal zoom system 78 and the imaging lens 74. In some examples, when the targets of the target area 58 are a linear array of point-like extended targets, no focusing lens 72 is needed. In some examples, when the targets of the target area 58 are a linear array of bar or oval- or elliptical-like targets, the focusing lens 72 may be utilized as needed.

The zoom system 75 with the afocal zoom configuration may include any suitable lens configuration. In some examples, the lenses of the afocal zoom configuration may comprise a first lens 80a to reduce a size of an image or light beam or ray 55 received from the target area, a second lens 80b configured to increase the size of the image or light beam or ray 55 received from the first lens 80a, and a third lens 80c configured to fix a size of the image or light beam or ray 55 received from the second lens 80b at a size at which the image or light beam or ray 55 is when the image or light beam or ray 55 contacts the third lens 80c. The first lens 80a and the second lens 80b may be axially adjustable relative to each other and to the third lens 80c. The third lens 80c may be at a fixed axial location relative to the first lens 80a and the second lens 80b, as depicted in FIGS. 7A-7C.

As depicted in FIG. 7A, the first lens 80a and the second lens 80b may be at a location adjacent one another, where the proximity of the first lens 80a to the second lens 80b and the spacing of second lens 80b from the third lens 80c may result in a magnification of an image or light beams or rays 55 received from the target area 58 at the first lens 80a. As depicted in FIG. 7B, the first lens 80a may be adjusted toward the target area 58 relative to a position of the first lens 80a in FIG. 7A and the second lens 80b may be adjusted toward the third lens 80c relative to a position of the second lens 80b in FIG. 7A, such that the resulting size of the image or light beam or ray 55 at the imaging lens 74 is equal to about the original image or light beam or ray 55 from the target area 58. As depicted in FIG. 7C, the first lens 80a may be adjusted away from the target area 58 relative to a position of the first lens 80a in FIG. 7B and closer to a position of the first lens 80a in FIG. 7A and the second lens 80b may be adjusted toward and to a location adjacent the third lens 80c relative to a position of the second lens 80b in FIG. 7B, such that the resulting size of the image or light beam or ray 55 at the imaging lens 74 is smaller than the original image or light beam or ray 55 from the target area 58.

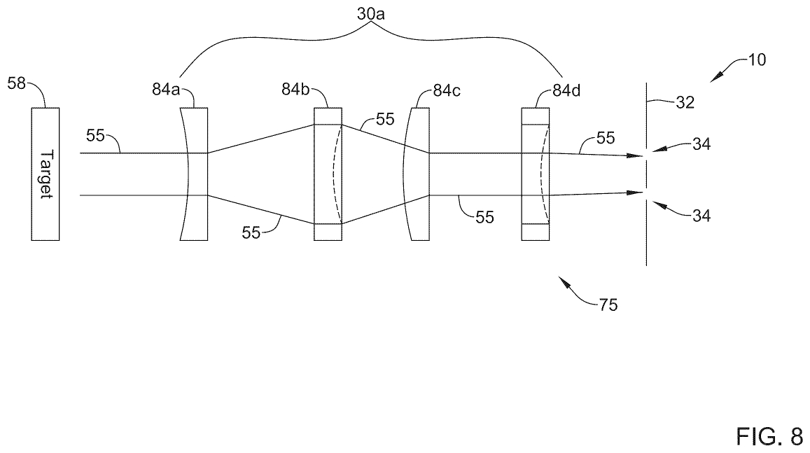

A similar function to the functions achieved by use of the zoom system 75 with the afocal zoom configuration discussed with respect to FIGS. 7A-7C may be achieved with focus tunable lens elements, such as tunable liquid lenses and/or other suitable focus tunable lens configurations. FIG. 8 depicts an illustrative configuration of the zoom system 75 with a tunable (e.g., electrically tunable) liquid lens zoom configuration.

As depicted in FIG. 8, the zoom system 75 with the tunable liquid lens configuration may include a first lens 84a at an axial location proximate to and fixed relative to the target area 58, where the first lens 84a may have a fixed configuration and may be configured to magnify the image or light beams or rays 55 from the target area 58. A second lens 84b of the tunable liquid lens zoom configuration may be at a fixed axial position between the first lens 84a and the opaque member 32, where the second lens 84b may be a liquid tunable lens configured to adjust how the lens bends the image or light beam or rays 55 received at the second lens 84b in response to a control signal. A third lens 84c of the tunable liquid lens zoom configuration may be at a fixed axial position between the second lens 84b and the opaque member 32, where the third lens 84c may have a fixed configuration and may be configured to reduce a size of the image or light beams or rays 55 from the target area relative to a size of the image or light beams or rays 55 received at the third lens 84c. A fourth lens 84d of the tunable liquid lens zoom configuration may be at a fixed axial position between the third lens 84c and the opaque member 32, where the fourth lens 84d may be a liquid tunable lens that may be configured to adjust how the lens bends the image or light beams or rays 55 received at the fourth lens 84d in response to a control signal. The image or light beams or rays 55 from the fourth lens 84d may be provided to the opaque member 32 and may create an interferogram at the light collection component 16 (not shown in FIG. 8) for processing, as discussed herein.

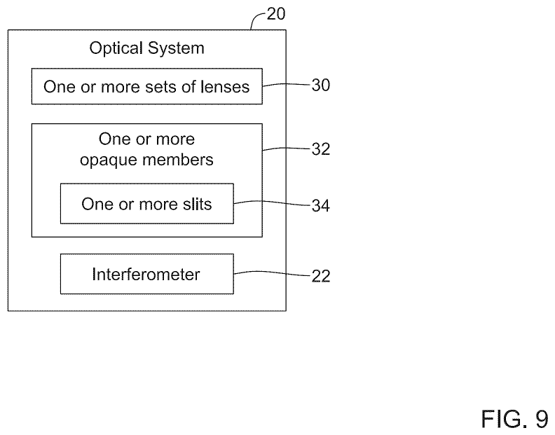

FIG. 9 depicts a schematic diagram of an illustrative configuration of the optical system 20, where the optical system 20 may include one or more interferometers 22. As discussed, the optical system 20 may include one or more sets of lenses 30 and one or more opaque members 32 including or defining one or more slits 34. In one example configuration, the optical system 20 may include a first set of lenses 30, a second set of lenses 30, an opaque member 32 having one or more slits 34 and positioned between the first set of lenses 30 and the second set of lenses 30, and the interferometer positioned entirely or at least partially on the same side of the opaque member 32 at which the second set of lenses 30 are located, where the first set of lenses 30 may be configured to provide light from the target area (e.g., provide an image or light beams or rays from the target area, such as an image of an array of reactants) to the opaque member 32 (e.g., to the one or more slits 34 in the opaque member 32) and the second set of lenses 30 may be configured to form or otherwise focus an interferogram from light passing through the interferometer 22 on a surface (e.g., a surface of the light collection component 16 and/or other suitable surface). Although not required, a housing (e.g., the housing 42 and/or other suitable housing) may be configured to house all of or at least a portion of the one or more sets of lenses 30 (e.g., the first set of lenses 30 and the second set of lenses 30), the one or more opaque members 32, and the interferometer 22.

The lenses of the one or more sets of lenses 30 may include any suitable type(s) of lenses configured (e.g., tuned and/or positioned) to receive, collect, and/or focus light from the target area (e.g., from one or more reactants of the reactant array 26) and direct the light to a surface (e.g., a surface of the one or more opaque members, a surface of a waveguide of the light collection component 16, a surface of the light or image sensor of the light collection component 16, and/or other suitable surface), as discussed herein or otherwise. The one or more sets of lenses may include any suitable configuration of lenses and may include sets of lenses 30 with a single lens, sets of lenses 30 with two or more lenses that may be similar to or different than one another, sets of lenses with one or more fixed location lenses, sets of lenses with one or more adjustable location lenses, sets of lenses with one or more adjustable focal points, and/or sets of lenses with other suitable lens configurations, as discussed herein or otherwise.

As discussed, the one or more opaque members 32 may have any suitable configuration configured to prevent light from the target area from passing between a first side of the opaque member(s) 32 (e.g., a side facing a direction of the target area) and a second side of the opaque member(s) 32 (e.g., a side facing a direction of the light collection component 16), except at the one or more slits. In some examples, an opaque member 32 of the one or more opaque members 32 may include a single slit 34 configured to receive light from target area such that the single slit 34 may create a single source of light from the target area for an interferometer 22 positioned between the one or more opaque members 32 and the light collection component 16.

The interferometer 22 may or may not include the opaque member 32. In some examples, the interferometer 22 may be configured to receive light from the target area, divide the received light into two or more beams, and result in the two or more beams interacting to create an interferogram on a surface (e.g., a surface of the light collection component 16). Example suitable configurations of interferometers 22 include, but are not limited to, a Michelson interferometer configuration, a Mach-Zehnder interferometer configuration, a birefringent crystal block interferometer configuration, a Wollaston prism interferometer configuration, a Rochon polarizing prism interferometer configuration, a Senarmont prism interferometer configuration, and/or other suitable interferometer configurations.

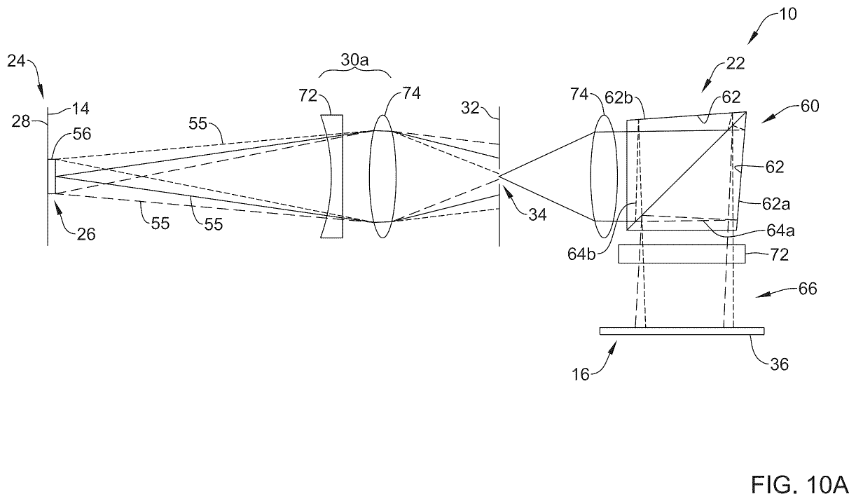

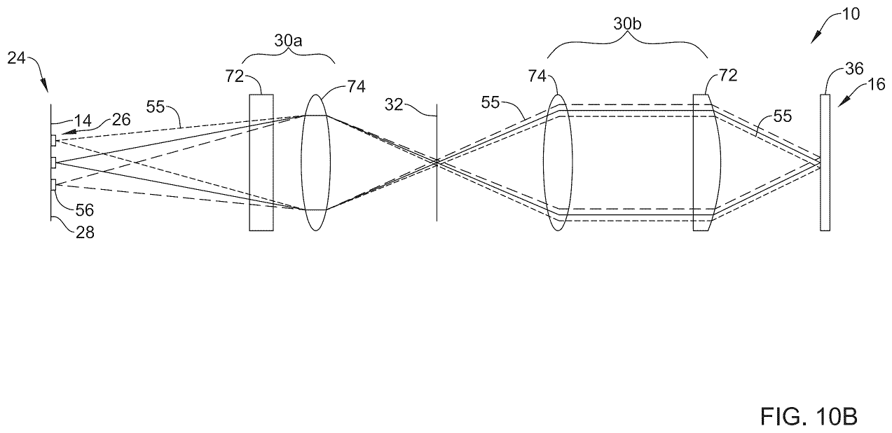

FIGS. 10-16 depict example illustrative configurations of the interferometer 22 positioned within the analysis system 10, where the analysis system 10 may enable Fourier transform hyperspectral imaging without a need for mechanical scanning of a target area. The analysis system 10 may be configured to analyze a target area with reactants 56 of the reactant array 26 supported by the substrate 28 as discussed herein, but use of the analysis system 10 with other suitable target areas is contemplated. Illustrative configurations of the analysis system 10 depicted in FIGS. 10-16 may include the first set of lenses 30a between the reactant array 26 and the opaque member 32, where the first set of lenses 30a may include a focusing lens 72 and an imaging lens 74 or other suitable configuration of one or more lenses as discussed herein or otherwise. In the configurations of the analysis system 10 depicted in FIGS. 10-16, the interferometer 22 may generally be positioned between the opaque member 32 and the light or image sensor 36 of the light collection component 16, where the opaque member 32 with a single slit 34 may act as a single light source for light from the reactant array 26 that is to be analyzed. A width of the single slit 34 may be selected to ensure spatial coherence of light from the reactant array 26 passing therethrough. In one example, the width of the single slit 34 may be less than a wavelength of the light passing therethrough, but other suitable widths (e.g., a few wavelengths or a few tens of wavelength) are contemplated. The configuration of the interferometer 22 in FIGS. 10-16 may achieve a similar spatial optical interference of light from the target area using an amplitude division approach as is achieved with the interferometer 22 using the opaque member 32 with the double slits 34.

FIGS. 10A and 10B depict a schematic side view and a schematic top view (e.g., views along orthogonal planes), respectively, of an illustrative configuration of the analysis system 10 including an illustrative interferometer 22 having a Michelson interferometer configuration that may produce interference fringes of an interferogram by splitting a light beam into two parts and then re-combining the two parts after the two parts have traveled different optical paths. The side view of FIG. 10A depicts the analysis system 10 in a folded, compact configuration facilitated by the use of the depicted interferometer 22. The top view of FIG. 10B depicts with the analysis system 10 in an unfolded configuration with the interferometer 22 omitted for clarity purposes.

FIG. 10A depicts the illustrative interferometer 22 positioned between an imaging lens 74 (e.g., a collimating spherical lens and/or other suitable imaging lens) and a focusing lens 72 (e.g., a cylinder lens) of a second set of lenses 30b. In some examples, a distance between the imaging lens 74 and the focusing lens 72 may be set to facilitate positioning the interferometer 22 between the imaging lens 74 and the focusing lens 72, but this is not required.

The illustrative interferometer 22 having the Michelson interferometer configuration may include a beam splitter/combiner 60 (e.g., a 50/50 beam splitter/combiner) configured as a cube or cube-like structure, with two, non-perpendicular mirrors 62 (e.g., mirrored surfaces and/or other suitable mirrors) that may be configured such that two light beams emitted from the interferometer 22 have a zero or near zero optical path length difference. When light from the imaging lens 74 passes through the beam splitter/combiner 60, the light may be split into a first light beam 64a that travels to a first mirror 62a and a second light beam 64b that travels to a second mirror 62b of the beam splitter/combiner 60 cube.

The first light beam 64a reflected back from the first mirror 62a may be combined with the second light beam 64b (e.g., where the first light beam 64a may be represented in FIG. 10A with smaller broken lines than the second light beam 64b) reflected back from the second mirror 62b to provide combined light beams 66 to the focusing lens 72 and/or to the light or image sensor 36. The combined light beams 66 may have a small crossing angle for the first light beam 64a and the second light beam 64b, which may interfere with one another and form an interferogram with spatial interference fringes on the light or image sensor 36. Further, the combined light beams 66 may be directed to travel in a direction perpendicular or at one or more other suitable angles relative to the light received at the beam splitter/splitter 60 from the single slit 34 in the opaque member 32, which may facilitate reducing an overall size of the analysis system 10 due to reducing a linear distance light needs to travel between the target area and the light or image sensor 36.

Although the beam splitter/combiner 60 takes the form of a cube in FIG. 10A, there may be other configurations that can be used to create a Michelson interferometer. For example, instead of using a beam splitter/combiner 60 having a cube configuration, a beam splitter/combiner 60 having a thin film or coating configuration with two mirrors 62 may be utilized to realize an interferometer 22 having a Michelson interferometer configuration, where a compensating plate may be positioned in one or both light paths between the beam splitter/combiner 60 and a respective mirror 62 to ensure an optical path length compensation of the separate light beams traveling different (e.g., non-equivalent) paths. Other suitable configurations of the beam splitter/combiner 60 configured to create an interferometer 22 having the Michelson configuration are contemplated.

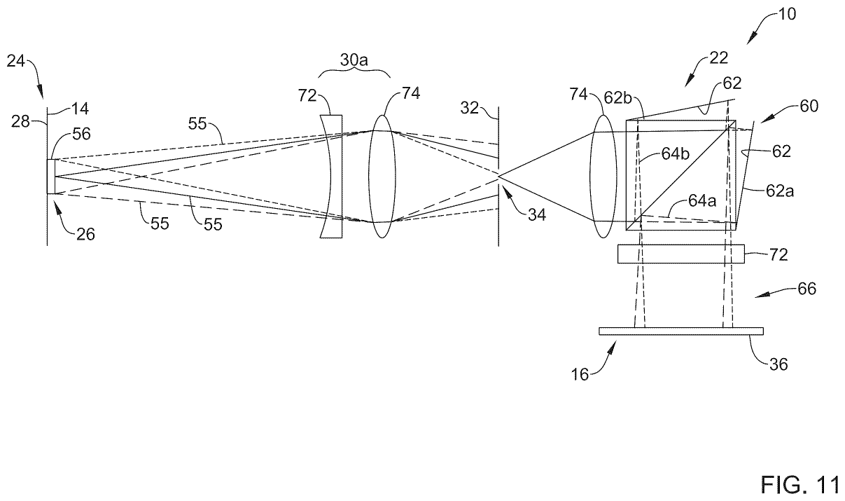

FIG. 11 depicts a schematic side view of an illustrative configuration of the analysis system 10 including an illustrative interferometer 22 having a Michelson interferometer configuration that may produce interference fringes of an interferogram by splitting a light beam into two parts and then re-combining the two parts after the two parts have traveled different optical paths. The side view of FIG. 11 depicts the analysis system 10 in a folded, compact configuration facilitated by the use of the depicted interferometer 22. A top view of the illustrative analysis system 10 depicted in FIG. 11 may be similar to the top view of the analysis system 10 depicted in FIG. 10B.

Similar to as depicted in FIG. 10A, FIG. 11 depicts the illustrative interferometer 22 positioned between an imaging lens 74 (e.g., a collimating spherical lens and/or other suitable imaging lens) and a focusing lens 72 (e.g., a cylinder lens) of a second set of lenses 30b. The illustrative interferometer 22 having the Michelson interferometer configuration depicted in FIG. 11 may include a beam splitter/combiner 60 (e.g., a 50/50 beam splitter/combiner) configured as a cube or cube-like structure, where two mirrors 62 may be located at or spaced from the cube of the beam splitter/combiner 60 and in light paths resulting from splitting a light beam or ray from the slit 34 in the opaque member 32 (e.g., from the target area). The two mirrors 62 may be non-perpendicular with respect to each other and may be configured such that two light beams emitted from the interferometer 22 have a zero or near zero optical path length difference. In such a configuration of the interferometer 22, when light from the imaging lens 74 passes through the beam splitter/combiner 60, the light is split into a first light beam 64a that travels to a first mirror 62a and a second light beam 64b that travels to a second mirror 62b.

Similar to as discussed with respect to FIG. 10A, a first light beam 64a reflected back from the first mirror 62a may be combined with a second light beam 64b (e.g., where the first light beam 64a may be represented in FIG. 11 with smaller broken lines than the second light beam 64b) reflected back from the second mirror 62b to provide combined light beams 66 to the focusing lens 72 and/or to the light or image sensor 36. The combined light beams 66 may have a small crossing angle of the first light beam 64a and the second light beam 64b, which may interfere with one another and form an interferogram with spatial interference fringes on the light or image sensor 36.

In some configurations, positioning the interferometer 22 between the imaging lens 74 and the focusing lens 72 may be optically beneficial. For example, the beam splitters/combiners 60 may be designed with a limited range of light beam or ray incident angles that work best for an intended beam split ratio over a wavelength range and thus, placing the interferometer 22 in a collimated light beam or ray path between the imaging lens 74 and the focusing lens 72 may ensure a narrower light beam or ray incidence angle range within the beam splitter/combiner 60. However, positioning the interferometer 22 between the imaging lens 74 and the focusing lens 72 is not required and other suitable configurations are contemplated.

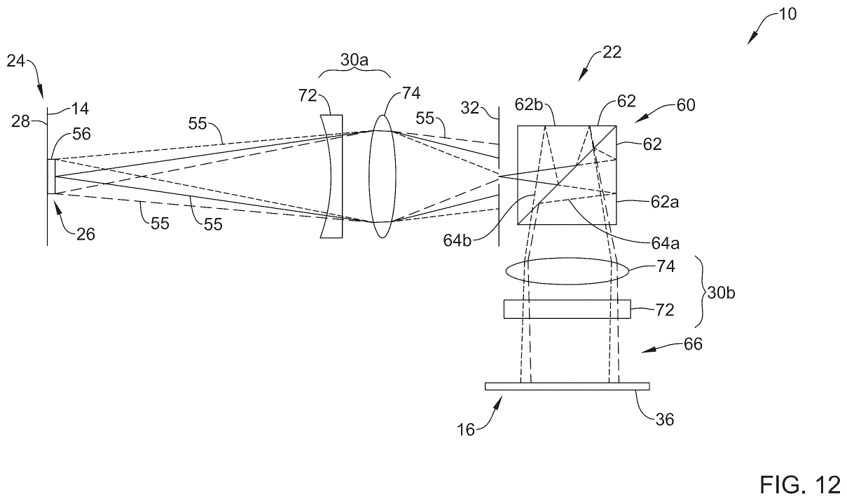

FIG. 12 depicts a schematic side view of an illustrative configuration of the analysis system 10 including an illustrative interferometer 22 having a Michelson interferometer configuration that may produce interference fringes of an interferogram by splitting a light beam into two parts and then re-combining the two parts after the two parts have traveled different optical paths, where the interferometer 22 is positioned between the opaque member 32 with a single slit 34 and the imaging lens 74 of the second set of one or more lenses 30b. The side view of FIG. 12 depicts the analysis system 10 in a folded, compact configuration facilitated by the use of the depicted interferometer 22. A top view of the illustrative analysis system 10 depicted in FIG. 12 may be the same as or similar to the top view of the analysis system 10 depicted in FIG. 10B.

As depicted in FIG. 12, the illustrative interferometer 22 having the Michelson interferometer configuration may include a beam splitter/combiner 60 (e.g., a 50/50 beam splitter/combiner) configured as a cube or cube-like structure, where two mirrors 62 may be located at or spaced from the cube of the beam splitter/combiner 60 and in light paths resulting from splitting of a light beam or ray from the slit 34 in the opaque member 32 (e.g., from the target area). The two mirrors 62 may be non-perpendicular with respect to each other and may be configured such that two light beams emitted from the interferometer 22 have a zero or near zero optical path length difference. In such a configuration of the interferometer 22, when light from the slit 34 in the opaque member 32 passes through the beam splitter/combiner 60, the light is split into a first light beam 64a that travels to a first mirror 62a and a second light beam 64b that travels to a second mirror 62b.

The first mirror 62a may be arranged to reflect the first light beam 64a backward in a direction toward the beam splitter/combiner 60. When the first light beam 64a hits the beam splitter/combiner 60 after reflecting off of the first mirror 62a, the first light beam 64a may be reflected toward the light or image sensor 36 and emerge from the interferometer 22 as a light beam propagating straight toward the light or image sensor from a virtual point source. When the first light beam 64a hits the imaging lens 74 and as long as the virtual point source is at a front focal plane of the imaging lens 74, the first light beam 64a may emerge from the imaging lens 74 as a collimated beam propagating toward the light or image sensor 36.

The second mirror 62b may be arranged to reflect the second light beam 64b in a direction toward the beam splitter/combiner 60 with a small angle. When the second light beam 64b hits the beam splitter/combiner 60 after reflecting off of the second mirror 62b, the second light beam 64b may be reflected toward the light or image sensor 36 slightly off axis angle and emerge from the interferometer 22 as a light beam at a slightly tilted angle relative to the first light beam 64a and from a virtual point source that may be transversely displaced from the virtual point source associated with the first light beam 64a. When the second light beam 64b hits the imaging lens 74 and as long as the virtual point source for the second light beam 64b is at a front focal plane of the imaging lens 74, the second light beam 64b may emerge from the imaging lens 74 with the first light beam 64a as collimated combined light beams 66 propagating with a slight crossing angles relative to one another and toward the light or image sensor 36.

When the first light beam 64a and the second light beam 64b overlap on the light or image sensor 36, spatial interference fringes of an interferogram may be formed on the light or image sensor 36. Therefore, the arrangement of the interferometer 22 being positioned adjacent to the single slit 34 of the opaque member 32 may function like an interferometer 22 having two slits 34 in the opaque member 32.

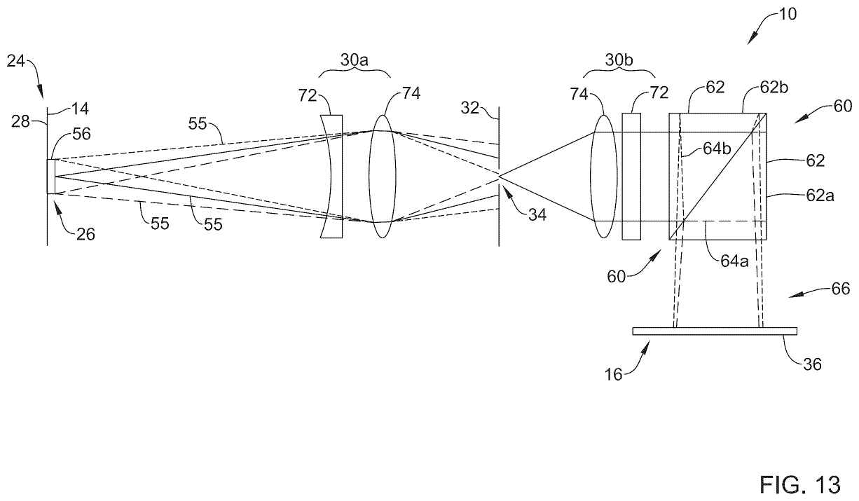

FIG. 13 depicts a schematic side view of an illustrative configuration of the analysis system 10 including an illustrative interferometer 22 having a Michelson interferometer configuration that may produce interference fringes of an interferogram by splitting a light beam into two parts and then re-combining the two parts after the two parts have traveled different optical paths, where the interferometer 22 is positioned between the second set of lenses 30b and the light or image sensor 36. The side view of FIG. 13 depicts the analysis system 10 in a folded, compact configuration facilitated by the use of the depicted interferometer 22.

As depicted in FIG. 13, the illustrative interferometer 22 may have the Michelson interferometer configuration depicted in FIG. 12. However, as the interferometer 22 is positioned between the focusing lens 72 of the second set of lenses 30b and the light or image sensor 36, the light beam or rays received at the interferometer may be collimated and focused and a first light beam 64a and a second light beam 64b emerging from the interferometer 22 as combined light beams 66 may have a crossing angle that produces spatial interference fringes of an interferogram on the light or image sensor 36.

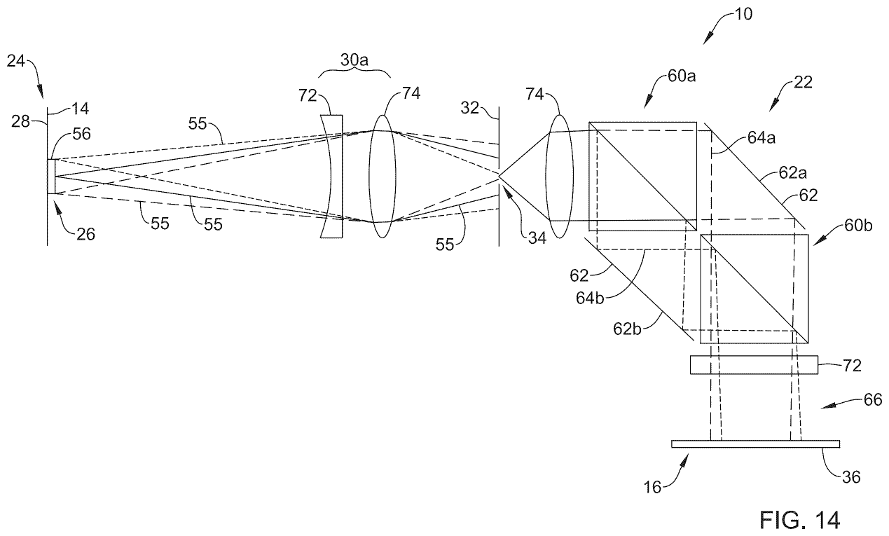

FIG. 14 depicts a schematic side view of an illustrative configuration of the analysis system 10 including an illustrative interferometer 22 having a Mach-Zehnder interferometer configuration that may produce interference fringes of an interferogram by splitting a light beam into two parts and then re-combining the two parts after the two parts have traveled different optical paths. The side view of FIG. 14 depicts the analysis system 10 in a folded, compact configuration facilitated by the use of the depicted interferometer 22.

Similar to as depicted in FIG. 10A, FIG. 14 depicts the illustrative interferometer 22 positioned between an imaging lens 74 (e.g., a collimating spherical lens and/or other suitable imaging lens) and a focusing lens 72 (e.g., a cylinder lens). The interferometer 22 having the Mach-Zehnder interferometer configuration may include a first beam splitter/combiner 60a (e.g., a first 50/50 beam splitter/combiner) and a second beam splitter/combiner 60b (e.g., a second 50/50 beam splitter/combiner), where a first mirror 62a and a second mirror 62b may be positioned between the first beam splitter/combiner 60a and the second beam splitter/combiner 60b. The first mirror 62a and the second mirror 62b may be non-parallel and positioned relative to one another such that beams exiting the second beam splitter/combiner 60b have a cross-angle that results in interference fringes of an interferogram on a light or image sensor 36 and a zero or near zero optical path length difference, where the interferogram may represent light from the target area.

In such a configuration of the interferometer 22, when light from the imaging lens 74 passes through the first beam splitter/combiner 60a, the light is split into a first light beam 64a that travels to a first mirror 62a and a second light beam 64b (e.g., where the first light beam 64a may be represented in FIG. 14 with larger broken lines than the second light beam 64b) that travels to a second mirror 62b. The first light beam 64a may be reflected off of the first the first mirror 62a to the second beam splitter/combiner 60b and the second light beam 64b may be reflected off of the second mirror 62a to the second beam splitter/combiner 60b, the first light beam 64a and the second light beam 64b may emerge from the second beam splitter/combiner 60b as combined light beams 66 with the first light beam 64a and the second light beam 64b having a crossing angle that creates spatial interference fringes of an interferogram on the light or imaging sensor 36.

Although the illustrative interferometer 22 depicted in FIG. 14 is located between the imaging lens 74 and the focusing lens 72, other suitable configurations are contemplated. For example, the illustrative interferometer 22 depicted in FIG. 14 may be located between the opaque member 32 having the single slit 34 and the imaging lens 74, between the focusing lens 72 and the light or image sensor 36, and/or at one or more other suitable locations.

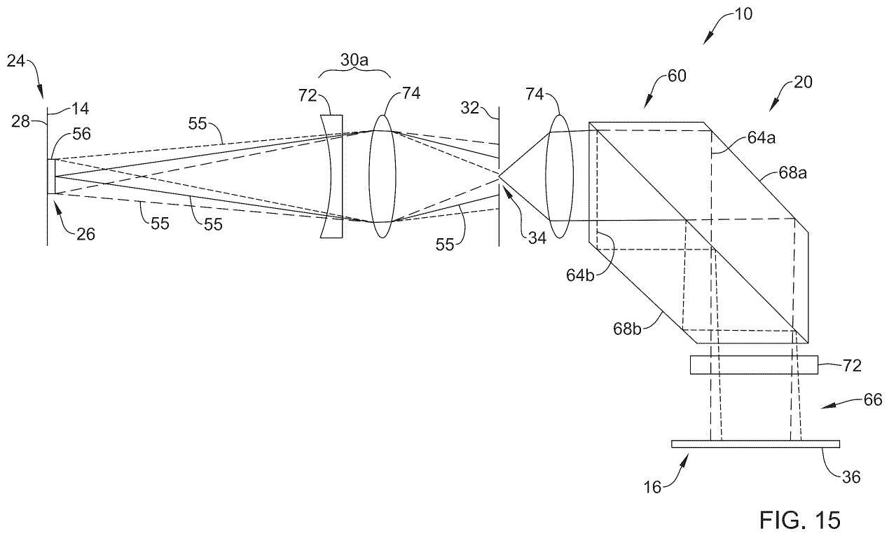

FIG. 15 depicts a schematic side view of an illustrative configuration of the analysis system 10 including an illustrative interferometer 22 formed from a block of prismatic glass or other suitable material that may produce interference fringes of an interferogram by splitting a light beam into two parts and then re-combining the two parts after the two parts have traveled different optical paths in a manner similar to how a Michelson interferometer configuration operates. The side view of FIG. 15 depicts the analysis system 10 in a folded, compact configuration facilitated by the use of the depicted interferometer 22.

FIG. 15 depicts the illustrative interferometer 22 positioned between an imaging lens 74 and a focusing lens 72. The interferometer 22 configured with a block of prismatic glass or other suitable material may include a beam splitter/combiner 60 (e.g., a 50/50 beam splitter/combiner), a first total internal reflection surface 68a, and a second total internal reflection surface 68b. Utilizing total internal reflection surfaces 68a, 68b rather than mirrors may remove a need for reflective coatings or metal surface of or on the interferometer 22. The first total internal reflection surface 68a and the second total internal reflection surface 68b may be non-parallel and positioned relative to one another such that light beams exiting the beam splitter/combiner 60 may have a crossing-angle that results in creating spatial interference fringes of an interferogram on a light or image sensor 36 and a zero or near zero optical path length difference, where the interferogram may represent light from the target area.

In such a configuration of the interferometer 22, when light from the imaging lens 74 passes through the beam splitter/combiner 60, the light may be split into a first light beam 64a that travels to a first total internal reflection surface 68a and a second light beam 64b (e.g., where the first light beam 64a may be represented in FIG. 14 with larger broken lines than the second light beam 64b) that travels to a second total internal reflection surface 68b. The first light beam 64a may be reflected off of the first total internal reflection surface 68a back to the beam splitter/combiner 60 and the second light beam 64b may be reflected off of the second total internal reflection surface 68b to the beam splitter/combiner 60, where the first light beam 64a and the second light beam 64b may emerge from the beam splitter/combiner 60 as combined light beams 66 with the first light beam 64a and the second light beam 64b having a crossing angle that creates spatial interference fringes of an interferogram on the light or imaging sensor 36.

Although the illustrative interferometer 22 depicted in FIG. 15 is located between the imaging lens 74 and the focusing lens 72, other suitable configurations are contemplated. For example, the illustrative interferometer 22 depicted in FIG. 15 may be located between the opaque member 32 having the single slit 34 and the imaging lens 74, between the focusing lens 72 and the light or image sensor 36, and/or at one or more other suitable locations.

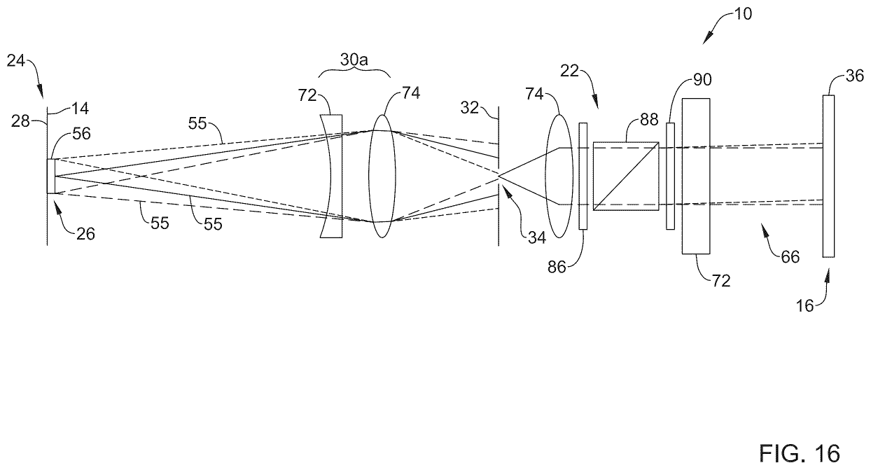

Further, amplitude division may be achieved via polarization splitting and recombining using prism(s) or prism combination structures. FIG. 16 depicts an analysis system 10 including an illustrative configuration of an interferometer 22 that may utilize polarization splitting and recombining using prism(s) or a combination of prism structures, where the interferometer 22 is positioned between the imaging lens 74 and the focusing lens 72 that are configured to receive light that has passed through the slit 34 of the opaque member 32. The interferometer 22 may include, among other suitable components, a polarizer 86, one or more prisms 88 (e.g., one or more birefringent crystal blocks, one or more Wollaston prisms, one or more Rochon polarizing prisms, one or more Senarmont prisms, etc.), and an analyzer 90. In some configurations, the polarizer 86 may be a first polarizer and the analyzer 90 may be configured as a second polarizer, but other suitable configurations are contemplated.

The polarizer 86 and the analyzer 90 may be any suitable type of polarizer. For example, the polarizer 86 and the analyzer 90 may be, but are not limited to, a thin film based linear polarizer, a wire-grid polarizer, a crystal base polarizer, a polarizing beam splitter so the formation mechanism includes linear polarization light absorption thin film, wire grid, optical crystal, and/or a polarization beam splitter, and/or other suitable type of polarizer. The polarizer 86 and the analyzer 90 may be a same type of polarizer as one another or different types of polarizers relative to the other, as desired.

In the illustrative configuration of the interferometer 22 depicted in FIG. 16, the interferometer 22 may receive a collimated beam from the imaging lens 74. The collimated beam may pass through the polarizer 86 having a pass-through axis oriented at 45 degrees relative to two optical axes of the prism 88 (e.g., two optical birefringent crystals of a Wollaston prism) such that half of the light beam received at the prism 88 is amplitude-divided after passing through the polarizer 86 and propagates and emerges from the prism 88 as a p-polarized light and the other half of the light beam propagates and emerges from the prism 88 as a s-polarized light. The p and s polarized light beams may have a crossing angle that is determined by a base angle of two constituent prisms (e.g., birefringent crystals and/or other suitable crystals) forming the prism 88 (e.g., the Wollaston prism, such as a small angle version). In some illustrative configurations, the analyzer 90 (e.g., oriented at a 45-degree angle relative to the two optical axes of the crystals forming the prism 88 and/or at one or more other suitable angles) may be arranged in a path of the two light beams emerging from the prism 88 to cause the two light beams emerging from the prism 88 combined light beams 66 to interfere with each other and produce a spatial interference pattern of an interferogram on the light or image sensor 36.

Although the prism 88 of the illustrative configuration the interferometer 22 discussed relative to FIG. 16 is described as being a Wollaston prism, other suitable birefringent crystal blocks with similar or different optical axis orientations and/or different shapes may be utilized to achieve the described splitting of a received light beam into two output beams having a crossing angle. For example, other birefringent crystal blocks suitable for use in or as the prism 88 include, but are not limited to, a small splitting-angle splitter comprising a birefringent crystal prism, a Rochon polarizing prism comprising two birefringent crystals, a Senarmont prism comprising two birefringent crystals, and/or suitable prisms utilizing one or more birefringent crystals and/or other suitable crystals.

Further, when utilizing birefringent crystals in the prism 88 of the interferometer 22, as discussed for example with respect to FIG. 16, an optical path length between two interfering beams emerging from the analyzer 90 may be affected by different refractive indices of the prism 88 such that when the beams recombine as the beams emerge from the analyzer 90, the beams may have different optical path lengths outside of a suitable coherence range. To address the different optical path lengths of the beams emerging from the interferometer 22, a compensating block of birefringent crystals (e.g., configured as a wave plate and/or other suitable configurations) may be used either before or after the analyzer 90 to bring the optical path length difference into the suitable coherence range.

Although the illustrative interferometer 22 depicted in FIG. 16 is located between the imaging lens 74 and the focusing lens 72, other suitable configurations are contemplated. For example, the illustrative interferometer 22 depicted in FIG. 16 may be located between the opaque member 32 having the single slit 34 and the imaging lens 74, between the focusing lens 72 and the light or image sensor 36, and/or at one or more other suitable locations.

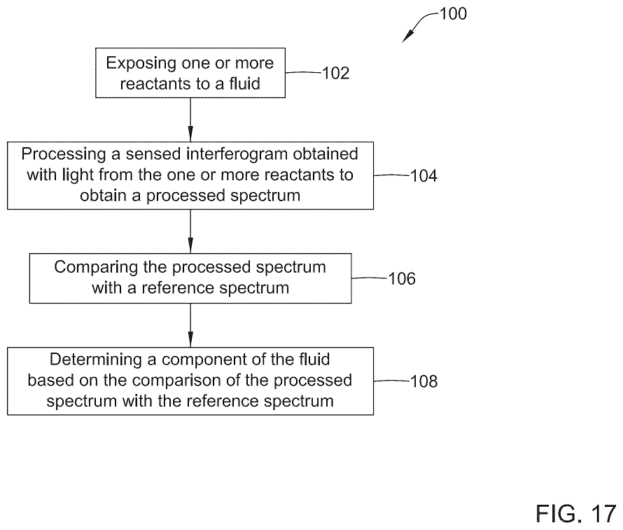

FIG. 17 depicts a method 100 that may facilitate performing a fluid analysis test on one or more fluids. The method 100 may include exposing 102 one or more reactants of a reactant array to a fluid. The reactants of the reactant array may be configured to react in a particular manner to one or more analytes and an interferogram of the reactants of the reactant array or a portion of the reactants of the reactant array may be sensed or captured by a light or image sensor of an analysis system. The configurations of the analysis system discussed herein and/or other suitable configurations of an analysis system may be utilized to sense or capture an interferogram of light from the reactant array. In some examples, the sensed or captured interferogram may be a Fourier transform of (e.g., a spatial frequency domain representation of) the optical spectrum of or from the reactant array

The sensed or captured interferogram may be processed 104 to obtain a processed spectrum of the light from the reactant array. As such, the sensed or captured interferogram may be processed by applying an inverse Fourier transform to the sensed or captured interferogram to obtain an optical spectrum of each reactant of the reactant array, an optical spectrum of the entire reactant array, and/or an optical spectrum of a desired portion of the reactant array. In some examples, reverse application of standard low or fast Fourier transform signal processing techniques may be utilized to obtain the processed spectrum from the reactant array based on the sensed or captured interferogram. In one example, the sensed or capture interferogram may be processed by subtracting a mid-point envelope profile of the sensed or captured interferogram from the sensed or captured interferogram, where the resulting interferogram may be transformed from a spatial frequency domain to a spectral domain to obtain the processed spectrum representing the optical spectrum of the reactant array.

In some cases, the processed spectrum representing the optical spectrum of the reactant array may be further processed to calibrate the processed spectrum based on spectrum from non-reactant portions of a substrate supporting the reactant array. For example, a reference spectrum obtained from a space between reactants of the reactant array may be obtained and used as a calibration reference, which may be used when obtaining the processed spectrum representing the optical spectrum of the reactant array. Any suitable calibration techniques using the reference spectrum may be utilized for calibrating the processed spectrum relative to the optics, sensors, and/or other configurations of the analysis system and/or relative to a configuration of the detecting component including the reactant array.

The processed spectrum may be compared 106 with one or more predetermined spectrums each associated with one or more fluid components of interest (e.g., analytes of interest or other fluids) and/or conditions of interest. Each of the predetermined spectrum may be representative of how one or more reactants of the reactant array may respond to being exposed to fluid components of interest or certain amounts of fluid components of interest. Although a single processed spectrum may be compared to predetermined spectrums, multiple spectrums obtained over time during the fluid analysis test may be compared to the predetermined spectrums, where the predetermined spectrums may or may not include sets of one or more time based (e.g., exposure time) spectrums for one or more fluid components of interest and/or conditions of interest.

Based on the comparison, a component (e.g., an analyte or other fluid) of the fluid tested in the fluid analysis test may be determined 108. For example, if one or more processed spectrums match one or more of the predetermined spectrums at a single time or over time, a component associated with a predetermined spectrum that matches the processed spectrum may be determined to be present in the fluid tested.

Although the method 100 describes a method of using the configurations of the analysis system in a fluid analysis test, the analysis system may be similarly utilized in one or more other suitable applications. For example, the analysis system described herein and/or other suitable analysis systems may be used in the method 100 or a similar method to analyze agricultural crop growth, antique objects authenticity (e.g., paintings, etc.), and/or other suitable conditions, where the exposure step may or may not be omitted.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

Unless otherwise expressly stated, it is in no way intended that any method or technique set forth herein is to be construed as requiring that its steps be performed in a specific order. This holds for any possible non-express basis for interpretation, including matters of logic with respect to arrangement of steps or operational flow, plain meaning derived from grammatical organization or punctuation, and the number or type of embodiments described in the specification

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A system for analyzing a target area, the system comprising:
an opaque member having one or more slits configured to be transverse to the target area;
one or more lenses configured to receive light from the target area; and
an image sensor configured to receive the light from the target area that has passed through the one or more slits and the one or more lenses.

2. The system of claim 1, wherein the one or more slits comprise a first slit and a second slit parallel to and spaced from the first slit.

3. The system of claim 1, wherein the one or more lenses comprise a focusing lens and an imaging lens configured to receive the light from the target area prior to the light passing through the one or more slits.

4. The system of claim 1, wherein the one or more lenses comprise a focusing lens and an imaging lens configured to receive the light from the target area after the light has passed through the one or more slits and before the light reaches the image sensor.

5. The system of claim 1, wherein the one or more lenses comprises:
a first set of one or more lenses configured to receive the light from the target area prior to the light passing through the one or more slits; and
a second set of one or more lenses configured to receive the light from the target area after the light has passed through the one or more slits and before the light reaches the image sensor.

6. The system of claim 1, further comprising:
an interferometer configured to receive the light from the target area after the light has passed through the one or more slits and before the light reaches the image sensor.

7. The system of claim 6, wherein the interferometer comprises:
one or more beam splitter/combiners;
a first mirrored surface; and
a second mirrored surface.

8. The system of claim 7, wherein the first mirrored surface is non-perpendicular relative to the second mirrored surface.

9. The system of claim 6, wherein the interferometer comprises:
a prism having a first total internal reflection surface and a second total internal reflection surface.

10. The system of claim 6, wherein the interferometer comprises:
a first polarizer;
a second polarizer; and
a beam splitter positioned between the first polarizer and the second polarizer.

11. The system of claim 1, wherein the target area includes an array of reactants, the light received at the one or more lenses and the image sensor is light from the array of reactants, and the system further comprises:
a substrate supporting the array of reactants; and
a controller in communication with the image sensor; and
wherein the controller is configured to identify a component of fluid in contact with the array of reactants based on the light from the array of reactants received at the image sensor.

12. An optical system for use in a fluid analysis system, the optical system comprises:
a first set of lenses;
a second set of lenses;
an opaque member having one or more slits therein and positioned between the first set of lenses and the second set of lenses, and
wherein the first set of lenses is configured to form an image of an array of reactants at the opaque member and the second set of lenses are configured to form an interferogram from light passing through the one or more slits on a surface.

13. The system of claim 12, wherein the one or more slits comprise a first slit and a second slit parallel to and spaced from the first slit.

14. The system of claim 12, wherein the first set of lenses comprises one or both of a focusing lens and an imaging lens configured to receive light from the array of reactants to form the image of the array of reactants on the opaque member.

15. The system of claim 12, wherein the second set of lenses comprise one or both of a focusing lens and an imaging lens configured to form the interferogram on the surface.

16. The system of claim 12, further comprising:
an interferometer configured to receive light from the array of reactants after the light has passed through the one or more slits.

17. The system of claim 12, further comprising:
a housing configured to house the first set of lenses, the second set of lenses, and the opaque member.

18. A hyperspectral imaging fluid analysis system comprising:
a substrate;
one or more reactants supported by the substrate;
an opaque member having one or more slits;
an image sensor configured to receive light from the one or more reactants that has passed through the one or more slits; and
a controller in communication with the image sensor.

19. The system of claim 18, wherein the controller is configured to identify a component of fluid in contact with the one or more reactants based on the light from the one or more reactants received at the image sensor.

20. The system of claim 18, wherein the one or more slits comprises a first slit and a second slit.
